(19) United States
(12) Patent Application Publication
(10) Pub. No.: US 2008/0280837 A1
(43) Pub. Date: Nov. 13, 2008

(54) Biologically Active Compounds with Anti-Angiogenic Properties

(76) Inventors: Judy Halliday, Chappel Hill (AU); Wim Meutermans, Toowong (AU); Gerald Tometzki, Manly West (AU); Tracie E. Ramsdale, Sunnybank (AU); Johannes Zuegg, Wynnum (AU); Bernd Becker, New Farm (AU); Craig Muldoon, Eight Mile Plains (AU); Declan McKeveney, Kagaroo Point (AU); Rajaratnam Premraj, Eight Mile Plains (AU); Glenn Condie, Kagaroo Point (AU)

Correspondence Address: MORRISON & FOERSTER LLP, 755 PAGE MILL RD, PALO ALTO, CA, 94304-1018, US

(21) Appl. No.: 11/547,343

(22) PCT Filed: Apr. 7, 2005
(86) PCT No.: PCT/AU2005/000506

§ 371 Date: Jul. 26, 2007

Related U.S. Application Data
(60) Provisional application No. 60/654,233, filed on Feb. 18, 2005.

(30) Foreign Application Priority Data
Apr. 8, 2004 (AU) 2004901887

(51) Int. Cl. A61K 31/7034; C12N 5/06; A61P 9/00

(52) U.S. Cl. 514/25; 435/375

(57) ABSTRACT

A method for inhibiting angiogenesis in a subject comprising administering to the subject at least one compound of General Formula (I), wherein the ring or any chiral center(s) may be of any configuration; Z is sulphur, oxygen, CH2, C(O), C(O)HN, NH, NRA or hydrogen, in the case where Z is hydrogen then R1 is not present, RA is selected from the set defined for R1 to R5, X and X′ are independently oxygen or nitrogen providing that at least one X of General Formula (I) is nitrogen, X or X′ may also combine independently with one of R1 to R5 to form an azide, R1 to R5 are independently selected from the following definition which includes but is not limited to H or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear, and R6 and R7 are hydrogen, or may combine to form a carbonyl function.

### FIELD OF THE INVENTION

The invention provides a class of biologically active compounds with anti-angiogenic properties.

### BACKGROUND OF THE INVENTION

Blood vessels form the largest network in the body and are the first organ to form in the developing embryo. The formation of new blood vessels is a complex, highly regulated process that is critically important for the development and homeostasis of an organism. Disruption to the regulation of the formation of new blood vessels contributes to malignant, inflammatory, immune and infectious disorders [Angiogenesis in health and disease, Carmeliet, P., Nature Medicine 2003, 9 (6), 653-660].

Recent attention has been focused on the “angiogenic switch” and its role in tumorigenesis. The complex stepwise progression towards malignancy has been well described for several types of cancer, in particular colon cancer, and is known to involve various genetic and epigenetic events leading to tumorigenesis. In addition to these events during transformation is the requirement for the induction of tumour vasculature, which allows the tumour to grow and spread. The induction of this vasculature is termed the “angiogenic switch” [Tumourigenesis and the Angiogenic Switch, Bergers, G. and Benjamin, L. E., Nature Reviews in Cancer 2003, 3, 401-410].

The classical model for the molecular regulation of angiogenesis involves a balance between pro-angiogenic molecules and anti-angiogenic molecules. There are at least twenty naturally occurring pro-angiogenic molecules identified to date including vascular endothelial growth factors (VEGFs), angiopoietins, fibroblast growth factors (FGFs), platelet-derived growth factors (PDGFs), epidermal growth factors (EGF's) and other growth factors and cytokines. To balance the scales there are at least thirty naturally occurring anti-angiogenic molecules identified to date. Somatostatin receptor subtypes have also been implicated in the inhibition of angiogenesis.

There is a continuing demand for the development of new and potent therapeutics for the treatment of cancer, inflammation, immune and infectious disorders. Inhibition of angiogenesis has proven to be a validated target in the treatment of these disorders. Compounds with somatostatin subtype 2 selectivity, subtype 2 & 3 dual selectivity and which inhibit subtypes 2, 3 and 5 have been previously identified by others as anti-angiogenic compounds. It is believed angiogenesis is associated with upregulation of the somatostatin 2 receptor [Pawlikowski, M., & Melen-Mucha G., Curr. Opin. in Pharmacol. 2004, 4, 608-613].

There is a need for compounds with improved anti-angiogenic activity.

### SUMMARY OF THE INVENTION

The invention provides compositions, methods, and kits for inhibition of angiogenesis, binding to somatostatin receptors, e.g., somatostatin receptor 5, and treatment of conditions for which inhibition of angiogenesis provides a therapeutic benefit.

It is a general object of the invention to provide compounds with anti-angiogenic properties, suitably, to arrest the development of malignant, inflammatory, immune and infectious disorders. In one aspect, the invention includes compounds described herein, and compositions comprising one or more of the compounds described herein, or tautomers, esters, solvates (e.g., hydrates), or pharmaceutically acceptable salts thereof. It is a further object of the invention to provide a pharmaceutical formulation comprising at least one compound as described herein or a tautomer, ester, solvate, or pharmaceutically acceptable salt thereof, together with one or more pharmaceutically acceptable carriers, diluents or excipients. In one embodiment, a pharmaceutical composition of the invention is provided as a pharmaceutically acceptable aqueous formulation, for example for parenteral administration, e.g., intravenously, intramuscularly. In some embodiments, a unit dose comprising one or more compounds of the invention is provided in a dry powder (e.g., lyophilized) form and reconstituted in a pharmaceutically acceptable carrier, such as a sterile aqueous formulation, prior to administration to an individual. In various embodiments, a pharmaceutical composition of the invention comprises one or more compounds of the invention and one or more pharmaceutical carriers, formulated for administration via a route selected from the group consisting of intravenous infusion or bolus, oral administration, intramuscular injection, suppository or pessiary, implant device, e.g., in the musculature or within a tumor, intraocular injection, transmucosal delivery, nasal delivery, or metered pump implant.

In another aspect, the invention provides a method of inhibition of angiogenesis, in vitro or in vivo. In one embodiment, the invention provides a method for inhibiting angiogenesis, comprising contacting a receptor associated with angiogenesis, for example a somatostatin receptor, e.g., somatostatin receptor subtype 5, with one or more compounds of the invention, wherein binding of said one or more compounds to said receptor inhibits angiogenesis.

In another embodiment, the invention provides a method for inhibiting angiogenesis, comprising contacting a sample comprising a blood vessel or a cell associated with formation of blood vessels with one or more compounds described herein, wherein contacting of said blood vessel or cell with said one or more compounds inhibits angiogenesis.

It is a further object of the invention to provide a method of treatment of a human or animal subject which method comprises administering to the human or animal subject an effective amount of a compound as described herein or a pharmaceutically acceptable salt thereof. In one embodiment, the invention provides a method of inhibiting angiogenesis in an individual in need thereof, comprising administering a pharmaceutical composition comprising a therapeutically effective amount of one or more compounds as described herein, or tautomers, esters, solvates, or pharmaceutically acceptable salts thereof, and a pharmaceutically acceptable carrier, to the individual. In some embodiments the invention provides a method for inhibiting growth of a tumor in an individual, comprising administering a pharmaceutical composition comprising a therapeutically effective amount of one or more compounds as described herein, or tautomers, esters, solvates, or pharmaceutically acceptable salts thereof, and a pharmaceutically acceptable carrier, to the individual. In some embodiments, the one or more compounds binds to somatostatin receptor subtype 5, thereby inhibiting angiogenesis.

### DETAILED DESCRIPTION OF THE INVENTION

The invention provides compounds and pharmaceutical compositions thereof that are useful for inhibition of angiogenesis both in vitro and in vivo, and kits comprising compounds of the invention. The invention also provides methods for inhibiting angiogenesis and methods for inhibiting tumor growth with compounds of the invention. The invention further provides methods for inhibiting activity of somatostatin receptors, e.g., somatostatin receptor subtype 5, and complexes comprising a compound of the invention bound to a somatostatin receptor. The invention also provides methods for inhibiting angiogenesis comprising binding of one or more compounds described herein to the somatostatin 5 receptor subtype.

We have identified compounds that interact in a biologically significant mariner, with somatostatin receptors. Surprisingly, compounds exhibiting their strongest interaction with the somatostatin 5 receptor subtype also exhibited potent anti-angiogenic activity. These compounds have now been shown to be anti-angiogenic it vitro, ex vivo and in vivo. A number of the compounds described herein have previously been described to interact with G protein coupled receptors (GPCRs) in PCT application no. PCT/AU2003/001347 (WO 2004/032940), which is incorporated by reference herein. As used herein, “biologically significant manner” refers to a binding interaction, e.g., a high affinity binding interaction, between a compound of the invention and a somatostatin receptor. Typically, such an interaction has an agonistic or antagonistic effect on receptor activity and/or an inhibitory effect on angiogenesis. Often, a compound of the invention interacts with somatostatin receptor subtype 5 with an IC50 of less than about 10 micromolar.

### Compositions

The invention provides compounds that are useful for binding to somatostatin receptors and for inhibition of angiogenesis, and pharmaceutical compositions thereof.

### Compounds of the Invention

In one aspect the invention provides for compounds of general formula I, that interact with one or more somatostatin receptors including somatostatin 5 in a biologically significant manner, thereby inhibiting angiogenesis,

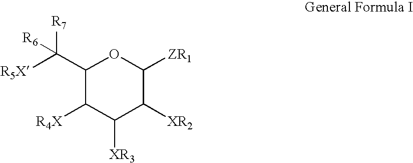

wherein the ring or any chiral center(s) may be of any configuration;
Z is sulphur, oxygen, CH₂, C(O), C(O)HN, NH, NR^{A} or hydrogen, in the case where Z is hydrogen then R₁ is not present, R^{A} is selected from the set defined for R₁ to R₅,
X and X′ are independently oxygen or nitrogen providing that at least one X of General Formula I is nitrogen, X or X′ may also combine independently with one of R₁ to R₅ to form an azide,
R₁ to R₅ are independently selected from the following definition which includes but is not limited to H or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear. Typical substituents include but are not limited to OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, any of which may optionally be further substituted, and
R₆ and R₇ are hydrogen, or may combine to form a carbonyl function.

In one embodiment the invention provides for compounds of general formula II that interact with one or more somatostatin receptors including somatostatin 5 in a biologically significant manner, thereby inhibiting angiogenesis,

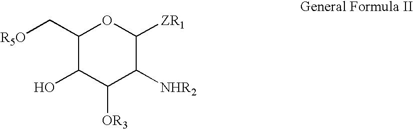

wherein R₁, R₂, R₃, R₅, and Z are defined as in General Formula I.

In another embodiment the invention provides for compounds of general formula III that interact with one or more somatostatin receptors including somatostatin 5 in a biologically significant manner, thereby inhibiting angiogenesis,

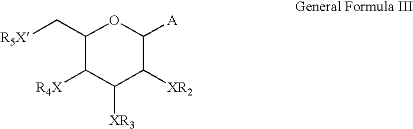

wherein A is defined as hydrogen, SR₁, or OR₁ where R₁ is defined as in General Formula I, and
X, X′, R₂, R₃, R₄, and R₅ are defined as in General Formula I.

In another embodiment the invention provides for compounds of General Formula IV that interact with one or more somatostatin receptors including somatostatin 5 in a biologically significant manner, thereby inhibiting angiogenesis,

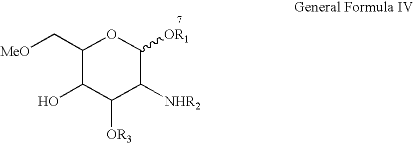

wherein R₁, R₂, and R₃ are defined as in General Formula I.

In another embodiment, the invention provides for compounds of General Formula V that interact with one or more somatostatin receptors including somatostatin 5 in a biologically significant manner, thereby inhibiting angiogenesis,

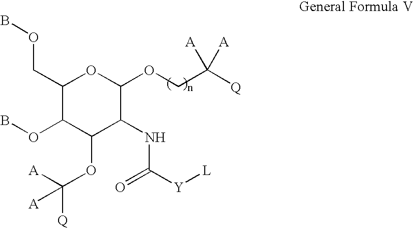

wherein the stereochemistry may be alpha or beta at the anomeric carbon, and may be axial or equatorial at the other pyranosyl ring carbons,
n is 0 or 1,
‘Y’ is selected from substituted or unsubstituted C1-C8 alkyl, hetero alkyl, cyclo-alkyl, aromatic or heterocyclic spacer, where typical substituents include but are not limited to nitro, chloro, fluoro, bromo, nitrite, carboxyl, —NH₂, —NHR, —NHB, C₁₋₃ alkyl, —OR, azido, —C(O)NH₂, —C(O)NHR, —C(O)N(R)₂, —N(R)C(O)R, —N(H)C(O)R, —CF₃, —SR, wherein R are typically independently selected from a substituted or unsubstituted alkyl, aryl or heterocyclic group,
L is selected from —NB₂, or guanidinium wherein B is defined as below, and additionally ‘Y’ and ‘L’ can combine to form a substituted or unsubstituted nitrogen containing heterocycle,
Q are independently selected from a substituted or unsubstituted monocyclic or bicyclic aromatic or hetero aromatic, where typical substituents are defined as for ‘Y’,
A are independently selected from hydrogen, chloro, fluoro or methyl, and
B are independently selected from H, methyl, ethyl, propyl.

In another embodiment the invention provides for compounds of General Formula VI that interact with one or more somatostatin receptors including somatostatin 5 in a biologically significant manner, thereby inhibiting angiogenesis,

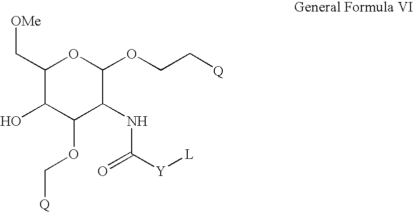

where Y, L, and Q are as defined in General Formula V.

In another embodiment the invention provides for compounds of General Formula VII that interact with one or more somatostatin receptors including somatostatin 5 in a biologically significant manner, thereby inhibiting angiogenesis,

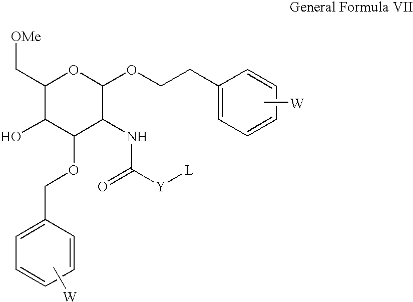

wherein, ‘W’ may represent mono-, di-, tri-, or tetrasubstitution and ‘W’ may be the same or different. Similarly, ‘W’ in combination with the aromatic ring, may represent a substituted or unsubstituted fused ring system which may be hetero-atomic or homo-atomic, and may be aromatic or aliphatic. Typical substituents include but care not limited to phenyl, C₁₋₄ alkyl, heterocycles, nitro, chloro, fluoro, bromo, nitrile, carboxyl, —NH₂, —NHR, —NR₂, C₁₋₃ alkyl, —OR, azido, —C(O)NH₂, —C(O)NHR, —C(O)N(R)₂, —N(R)C(O)R, —N(H—)C(O)R, —CF₃, —SR, wherein R are typically independently selected from a substituted or unsubstituted alkyl, aryl or heterocyclic group,
and where Y and L are as defined in General Formula V.

In a further embodiment the invention provides for compounds of General Formula VIII that interact with one or more somatostatin receptors including somatostatin 5 in a biologically significant manner, thereby inhibiting angiogenesis,

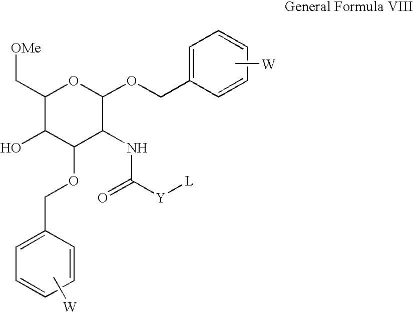

wherein, W, L and Y are as defined above;

In a further embodiment the invention provides for compounds of General Formula IX that interact with one or more somatostatin receptors including somatostatin 5 in a biologically significant manner, thereby inhibiting angiogenesis,

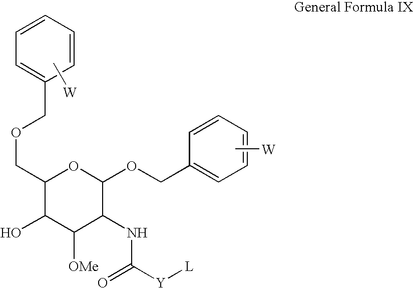

wherein, W, L and Y are as defined above;

In a further preferred embodiment the invention provides for compounds of General Formula X that interact with one or more somatostatin receptors including somatostatin 5 in a biologically significant manner, thereby inhibiting angiogenesis,

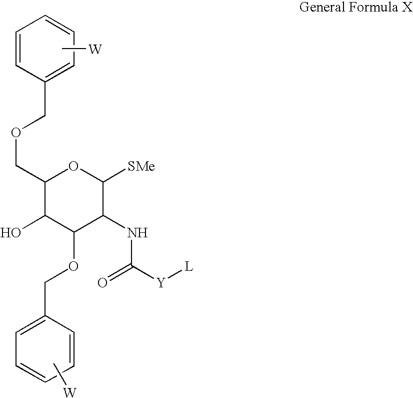

wherein, W, L and Y are as defined above.

In all embodiments described above, where a group may be optionally or further substituted, the possible substituents are selected from the group consisting of OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl and thioheteroaryl. In a preferred embodiment, the substituents are selected from the group consisting of OH, NO, NO₂, NH₂, N₃, halogen, CF₃, CHF₂, CH₂F, nitrile, alkoxy, amidine, guanidiniums, carboxylic acid, carboxylic acid ester, carboxylic acid amide, aryl, heteroaryl, aminoalkyl, aminodialkyl, aminotrialkyl, aminoacyl, hydroxamate, hydroxamic acid and thioalkyl.

### Pharmaceutical Compositions

In another aspect, the invention provides pharmaceutical compositions comprising any of the compounds described herein, or tautomers, esters, solvates, or pharmaceutically acceptable salts thereof, and a pharmaceutically acceptable carrier.

in some embodiments, a pharmaceutically acceptable aqueous formulation is provided that is suitable for parenteral administration, such as, for example, intravenous injection. For preparing such an aqueous formulation, methods well known in the art may be used, and any pharmaceutically acceptable carriers, diluents, excipients, stabilizers, or other additives normally used in the art may be used.

A pharmaceutical composition for parenteral administration includes a physiologically acceptable diluent such as deionized water, physiological saline, 5% dextrose, water miscible solvent (e.g., ethyl alcohol, polyethylene glycol, propylene glycol, etc.), non-aqueous vehicle (e.g., oil such as corn oil, cottonseed oil, peanut oil, and sesame oil), or other commonly used diluent. The formulation may additionally include a solubilizing agent such as polyethylene glycol, polypropylene glycol, or other known solubilizing agent, buffers for stabilizing the solution (e.g., citrates, acetates, and phosphates) and/or antioxidants (e.g., ascorbic acid or sodium bisulfite). (See, for example, U.S. Pat. No. 6,143,739.) Other suitable pharmaceutical carriers and their formulations are described in “Remington's Pharmaceutical Sciences” by E. W. Martin. As is known in the art, pharmaceutical preparations of the invention may also be prepared to contain acceptable levels of particulates (e.g., particle-free) and to be non-pyrogenic (e.g., meeting the requirements of an injectable in the U.S. Pharmacopeia).

In some embodiments, pharmaceutical compositions of the invention comprise one or more compounds described herein and a pharmaceutically acceptable carrier, suitable for administration via parenteral administration, e.g., intravenous, intramuscular, subcutaneous. In various embodiments, pharmaceutical compositions of the invention comprise one or more compounds described herein and a pharmaceutically acceptable carrier, suitable for administration via a route selected from the group consisting of intravenous infusion or bolus injection, oral administration, intramuscular injection, suppository or pessiary, implant device, e.g., in the musculature or within a tumor, intra-ocular injection, transmucosal delivery, nasal delivery, or metered pump implant.

Complexes with Somatostatin Receptors

In another aspect, the invention provides a complex between a receptor, e.g., a receptor that is involved in angiogenesis, and a bound compound as described herein. For example, a complex of the invention may comprise a compound described herein and a somatostatin receptor. In one embodiment, the complex comprises a compound described herein, and a somatostatin receptor, for example, somatostatin receptor subtype 5. In one embodiment, the complex comprises “compound 1” (described infra) and somatostatin receptor subtype 5.

### Methods of the Invention

### Methods for Inhibiting Angiogenesis

In one aspect, the invention provides a method of inhibition of angiogenesis, in vitro or in vivo. As used herein, “inhibition of angiogenesis” refers to inhibition of formation of new blood vessels; for example, inhibition of the proliferation, migration, and/or differentiation of cells associated with the growth and/or formation of new blood vessels (e.g., endothelial cells, endothelial progenitor cells, bone marrow cells, smooth muscle cells). Inhibition of angiogenesis may be assessed by methods that are well known in the art, including those described in the Examples herein. Examples of assays for inhibition of angiogenesis include cell proliferation, migration, and differentiation assays, the rat aortic ring assay, chicken chorioallantoic membrane assay, the in vivo matrigel plug assay, and other implant assays. These assays are described in “Angiogenesis Assays: A Critical Overview” [Auerbach, R., et al. (2003) Clinical Chemistry 49(1):32-40] and references therein.

In one embodiment, the invention provides a method for inhibiting angiogenesis, comprising contacting a receptor associated with angiogenesis, for example a somatostatin receptor, e.g., somatostatin receptor subtype 5, with one or more compounds of the invention, wherein binding of said one or more compounds to said receptor inhibits angiogenesis. As used herein, “binding” of a compound of the invention refers to a specific binding interaction between the compound and the receptor, such that the compound acts as an agonist or antagonist of the receptor. Generally, the interaction between the compound and the receptor is of high affinity. In some embodiments, the IC50 of a compound of the invention is less than about 10 micromolar, 1 micromolar, or 0.5 micromolar at the somatostatin 5 receptor. As used herein, “IC50” refers to the concentration of compound required to displace 50% of the native receptor ligand. Receptor binding may be assessed according to a number of well known techniques in the art, including radio-ligand binding assails, cell based assays, and signal transduction pathway assays, as described in Current Protocols in Pharmacology Ed., Enna, S. J., et al., published by John Wiley & Sons.

In another embodiment, the invention provides a method for inhibiting angiogenesis, comprising contacting a sample comprising a blood vessel or a cell associated with formation of blood vessels (e.g., endothelial cells, endothelial progenitor cells, bone marrow cells, smooth muscle cells) in vitro or in vivo with one or more compounds described herein, wherein contacting of said blood vessel or cell with said one or more compounds inhibits angiogenesis. In some embodiments, angiogenesis is inhibited at least about 10, 20, 30, 40, 50, 60, 70, 80, 90, or 95% in comparison to a control sample which has not been contacted with the one or more compounds.

### Methods of Treatment

The invention provides methods of treatment comprising administering one or more compounds of the invention to an individual in need of treatment for a condition for which inhibition of angiogenesis is therapeutically beneficial.

In one embodiment, the invention provides a method of inhibiting angiogenesis in an individual in need thereof, comprising administering a pharmaceutical composition comprising a therapeutically effective amount of one or more compounds as described herein, or tautomers, esters, solvates, or pharmaceutically acceptable salts thereof, and a pharmaceutically acceptable carrier, to the individual. In some embodiments, angiogenesis is inhibited at least about 10, 20, 30, 40, 50, 60, 70, 80, 90, or 95% in comparison to an individual to whom the pharmaceutical composition has not been administered.

In some embodiments the invention provides a method for inhibiting growth of a tumor in an individual, comprising administering a pharmaceutical composition comprising a therapeutically effective amount of one or more compounds as described herein, or tautomers, esters, solvates, or pharmaceutically acceptable salts thereof, and a pharmaceutically acceptable carrier, to the individual. In some embodiments, tumor growth is inhibited at least about 10, 20, 30, 40, 50, 60, 70, 80, 90, or 95% in comparison to an individual to whom the pharmaceutical composition has not been administered. In some embodiments, one or more additional therapeutic compounds is administered simultaneously or sequentially, in a combination therapy, for example, one or more chemotherapeutic substances. In one embodiment, one or more chemotherapeutic agents of the taxoid class of anti-tumor compounds, e.g., paclitaxel, docetaxel, is administered simultaneously or sequentially with one or more compounds described herein. In other embodiments, 5-fluorouracil, methotrexate, or a platinum drug, e.g., cisplatin, carboplatin, oxaliplatin, is administered simultaneously or sequentially with one or more compounds described herein. In one embodiment, the chemotherapeutic agent(s) and the compound(s) described herein act synergistically to inhibit tumor growth.

As used herein, “individual” refers to a vertebrate, typically a mammal, often a human.

As used herein, “therapeutically effective amount” refers to the amount of a compound that will render a desired therapeutic outcome (e.g., inhibition of angiogenesis or reduction of tumor growth). A therapeutically effective amount may be administered in one or more doses. A therapeutically effective dosage of a compound described herein is sometimes about 1 μg/kg to about 100 mg/kg, sometimes about 50 μg/kg to about 25 mg/kg.

Administration may be via any route suitable for the condition being treated. For example, administration may be parenteral, e.g., intravenous (infusion or bolus injection), intramuscular, subcutaneous, or may be via suppository or pessiary, implantable device, for example intramuscular or within a tumor, intra-ocular injection, transmucosal, transdermal, or nasal administration, or via a metered pump implant.

Compounds described herein are useful for treatment of conditions for which inhibition of angiogenesis is therapeutically beneficial. For example, compounds described herein may be used for treatment of type I or type II diabetes mellitus, including complications thereof, e.g., angiopathy, diabetic proliferative retinopathy, diabetic macular edema, nephropathy, neuropathy, neuropathy and dawn phenomenon, and other metabolic disorders related to insulin or glucagon release, e.g., obesity, for example morbid obesity or hypothalamic or hyperinsulinemic obesity. Compounds described herein may also be used for the prevention or treatment of angiogenesis and inflammatory disorders including inflammatory eye diseases, macular edema, e.g., cystoid macular edema, idiopathic cystoid macular edema, exudative age-related macular degeneration, choroidal neovascularization related disorders and proliferative retinopathy. The compounds described herein may also be used in the treatment of enterocutaneous and pancreaticocutaneous fistula, irritable bowel syndrome, inflammatory diseases, e.g., Grave's disease, inflammatory bowel disease, psoriasis or rheumatoid arthritis, polycystic kidney disease, dumping syndrome, watery diarrhea syndrome, AIDS-related diarrhea, chemotherapy-induced diarrhea, acute or chronic pancreatitis, gastrointestinal bleeding, e.g., variceal oesophagial bleeding. Compounds described herein may also be used in the treatment of tumors and malignant cell proliferative diseases, for example, gastrointestinal hormone secreting tumors (e.g., GEP tumors, for example vipomas, glucagonomas, insulinomas, carcinoids), lymphocyte malignancies, e.g., lymphomas, leukemias, hepatocellular carcinoma, colon and bowel, liver, breast, prostate, lung, stomach, pancreas, or other GI tract cancers.

### Kits

The invention also provides kits for use in methods of the invention. The kits include one or more compounds described herein. A kit may include a pharmaceutical composition as described herein, for example including at least one therapeutically effective dose of at least one compound of the invention, and optionally instructions for use, for example, instructions providing information to a health care provider regarding usage in a method of the invention as described above. Instructions may be provided in printed form or in the form of an electronic medium such as a floppy disc, CD, or DVD, or in the form of a website address where such instructions may be obtained. In some embodiments, the kit comprises a compound described herein as a sterile aqueous pharmaceutical composition or as dry powder (e.g., lyophilized) composition.

Suitable packaging is provided. As used herein, “packaging” refers to a solid matrix or material customarily used in a system and capable of holding within fixed limits a composition suitable for administration to an individual. Such materials include glass and plastic (e.g., polyethylene, polypropylene, and polycarbonate) bottles, vials, paper, plastic, and plastic-foil laminated (envelopes and the like. If e-beam sterilization techniques are employed, the packaging should have sufficiently low density to permit sterilization of the contents.

Kits may also optionally include equipment for administration of a pharmaceutical composition, such as, for example, syringes or equipment for intravenous administration, and/or a sterile solution, e.g., a diluent, for preparing a dry powder (e.g., lyophilized) composition for administration.

The following Examples are intended to illustrate, but not limit, the invention.

### EXAMPLES OF THE INVENTION

### Example 1

### Ex Ovo Determination of Antiangiogenic Effects Using the Early Chicken Embryo Chorioallantoic Membrane (EarlyCAM)

“Compound 1” was assayed to determine its anti-angiogenic characteristics ex vivo according to a previously published method [A novel early chorioallantoic membrane assay demonstrates quantitative and qualitative changes caused by antiangiogenic substances, Hazel, J Lab Clin Med, 2003, 141, 217-28].

### Vein Diameter

Vein diameter was included as a measure of vessel growth. Where there was more than one major vein branch, the diameters of both were added together to give a total vein diameter. In the control CAM there were two well developed vein branches of similar diameter. In contrast, in the treated CAM there was a single major vein branch. Total vein diameter is 239 pixels in the control CAM, and only 107 pixels in the treated CAM.

### Octeotride

As a comparison, octeotride was tested in the earlyCAM assay. At a dose of 10 nmol octeotride did not appear to have any significant effect on the CAM vasculature.

### General Results

When “compound 1” was applied to the CAM, there were dose responsive reductions in CAM growth and vasculature. The effects on CAM growth and vessel parameters were expressed as pixel measures (Table 1) and also as a percentage of the vehicle treated control group (FIG. 1). Vein lengths were reduced at all dose levels of “compound 1”, both in pixel and percentage terms, with a reduction to 67% of control at 5 nmol (p<0.05). In contrast the artery lengths were not reduced as much, with a maximal reduction to 86% of control at 1 nmol. When artery and vein lengths were combined to give total vessel length there was a significant reduction at the 5 nmol level (FIG. 1; p<0.05). A strong trend to reduced CAM growth with increasing doses of “compound 1” was also present. Hence, when vessel lengths were expressed relative to the CAM size, relative vessel lengths were not significantly changed with “compound 1” treatment.

Vein diameter was also reduced in a dose dependent manner, from 215 pixels in the vehicle control group to 157 pixels in the 5 nmol group (p=0.057). This represents an approximately 25% reduction in vein diameter in the 5 nmol group versus control.

In the vehicle control the vessels were well developed and regularly organised. Following treatment with 1 nmol of “compound 1” there was some distortion of the CAM, but the vessels were still reasonably well developed. However, in the CAM treated with 5 nmol of “compound 1” there was a single attenuated major vein branch and far fewer vessels. In the higher magnification image the avascular areas between the vessels were apparent, and the major vessel branches are relatively thin compared with the control CAM.

TABLE 1

| | |
|---|---|
| | 1 |
| 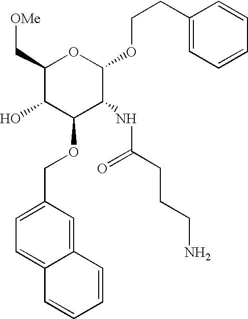 | |

Effects of “compound 1” on vessel parameters in the earlyCAM assay.
Mean ± SEM; n = 8.

| | Vehicle | 0.2 nmol | 1 nmol | 5 nmol |
|---|---|---|---|---|
| CAM increase | 11.1 ± 0.7 | 9.9 ± 0.9 | 9.9 ± 0.8 | 8.9 ± 0.8 |
| (fold) | | | | |
| Vein length | 2158 ± 158 | 1628 ± 183 | 1625 ± 166¹ | 1429 ± 141¹ |
| (pixels) | | | | |
| Aretry length | 1922 ± 156 | 1870 ± 109 | 1544 ± 214 | 1510 ± 196 |
| (pixels) | | | | |
| Total vessel | 4080 ± 289 | 3498 ± 222 | 3169 ± 341 | 2939 ± 292¹ |
| length (pixels) | | | | |
| Relative vein | 42.8 ± 1.9 | 36.9 ± 3.4 | 36.5 ± 2.4 | 35.1 ± 2.6 |
| length* | | | | |
| Relative artery | 38.0 ± 1.4 | 43.0 ± 3.7 | 34.7 ± 4.7 | 36.4 ± 3.0 |
| length* | | | | |
| Relative total | 80.8 ± 2.1 | 79.9 ± 5.8 | 71.3 ± 6.3 | 71.6 ± 3.7 |
| vessel length* | | | | |
| Vein diameter^{a} | 215 ± 9 | 191 ± 10 | 187 ± 15 | 157 ± 23 |
| (pixels) | | | | |

*Relative vessel length = absolute vessel length (pixels)/CAM area (pixels)
¹p < 0.05 vs vehicle
^{a}p = 0.057

### Example 2

### In Vivo Pharmacokinetic Evaluation of “Compound 1” after i.v. and p.o. Administration to Rats

### Experimental Conditions

Intravenous infusion of “compound 1” (2 mg/Kg) over 5 minutes to two rats and arterial blood sampled up to 24 hours.

Oral administration of “compound 1” (25 mg/Kg as HPMC suspension) via oral gavage to two rats and arterial blood sampled up to 24 hours.

Plasma concentrations of “compound 1” determined by MS (LOQ<0.01 μM)

Calculations:

$${CL}_{total} = \frac{{Dose}_{IV}}{{AUC}_{IV}} \qquad {V}_{d β} = \frac{{CL}_{total}}{β} BA ( \% ) = \frac{{AUC}_{oral} * {Dose}_{IV}}{{AUC}_{IV} * {Dose}_{oral}}$$

CLₜₒₜₐₗ=total plasma clearance after IV administration
V_{dβ}=volume of distribution during the elimination phase after IV administration
BA=oral bioavailability
AUC_{IV}=area under the plasma concentration versus time profile from time zero to infinity after IV administration
AUCₒᵣₐₗ=area under the plasma concentration versus time profile from time zero to infinity after oral administration
β=terminal elimination rate constant after IV administration

### Summary

Following an I.V. dose, the elimination half-life of “compound 1” was approx 4.6 h. The clearance and volume of distribution values were 8.20 mL/min/Kg and 3.30 L/Kg, respectively. The bioavailability of “compound 1” following oral dosing was approximately 5.2%. This is based on the AUC from 0 to 480 min.

TABLE 2
Pharmacokinetic parameters following I.V. and oral
administration of “compound 1” to rats.

| | Rat 1 | Rat 2 | | Rat 3 | Rat 4 | |
|---|---|---|---|---|---|---|
| Parameter | IV | IV | Mean ± SD | PO | PO | Mean ± SD |
| Measured | 2.66 | 2.67 | 2.67 ± 0.01 | 27.17 | 29.64 | 28.41 ± 1.75 |
| Dose | | | | | | |
| (mg/Kg) | | | | | | |
| Cₘₐₓ (μM) | 30.26 | 33.48 | 31.87 ± 2.27 | 6.95 | 2.94 | 4.95 ± 2.84 |
| Tₘₐₓ (min) | — | — | — | 20 | 20 | 20 |
| t_{1/2} (h) | 4.45 | 4.76 | 4.60 ± 0.22 | n.d. | n.d. | n.d. |
| Clₜₒₜₐₗ^{a} | 7.53 | 8.86 | 8.20 ± 0.94 | — | — | — |
| (ml/min/ | | | | | | |
| Kg) | | | | | | |
| V_{dB} | 2.90 | 3.69 | 3.29 ± 0.56 | — | — | — |
| (L/Kg) | | | | | | |
| BA^{b} (%) | — | — | — | 4.95 | 5.38 | 5.16 ± 0.30 |

^{a}Total plasma clearance
^{b}Oral BA calculated using AUC₀₋₄₈₀.
n.d. not determined

### Example 3

### In Vivo Efficacy Evaluation of “Compound 1” in a Nude Mouse Model of Human PC-3 Prostate Tumours

Initial Maximum Tolerated Dose (MTD) studies were done in male nude mice to determine the appropriate intravenous dosing regimen for the PC-3 human prostate tumour model. A range of doses between 0 and 50 mg/Kg for 28 days (qdx28) were tested. Mice were randomized into groups with 5 animals per group including vehicle control. Animals were weighed twice weekly starting on day one and observed daily for adverse reactions or toxicity due to the agent. MTD studies determined the selection of 20 mg/kg and 35 mg/kg for intravenous dosing once per day for 28 days in the PC-3 human prostate tumour xenograft model.

Male nude mice (nu/nu) between 5 and 6 weeks of age weighing approximately 25 g were implanted subcutaneously (s.c) by trocar with fragments of PC-3 human tumour carcinomas harvested from s.c growing tumours in host mice. PC-3, is a metastatic human prostate adenocarcinoma cell line originating from a 62 year old Caucasian male. When tumours reached approximately 36 mm³ in size animals were pair matched into treatment and control groups with 10 mice in each group. Each mouse was tagged and followed individually throughout the experiment.

“Compound 1” was administered i.v. in a saline vehicle from day one. Vehicle control group animals were administered saline i.v. There were two treatment groups, one group received 20 mg/kg of “compound 1” i.v. and the second group received 35 mg/kg of “compound 1” i.v. These 3 groups were treated daily for 29 days. A fourth positive control group of animals were given the standard chemotherapeutic agent Taxotere® i.v on days 1, 3 and 5 of the study.

Mice were weighed twice weekly and tumour measurements were obtained using calipers twice weekly. Collection of measurements started on day 1. Tumour measurements were converted into tumour volume (mm³) using the standard formula (W²×L)×0.52.

At the end of the treatment period the mice were weighed and sacrificed. Each tumour was excised and weighed and a mean actual tumour weight (mg) per group was calculated along with the mean actual volume (mnm³). Mice having a tumour with less volume than on day 1 were classified as having partial tumour regression. Mean tumour regression was determined using the formula [1−(mean actual tumour weight_{FINAL}/mean tumour weight_{DAY 1})×100%].

Tumour growth inhibition (TGI) was calculated for each group containing treated animals that did not demonstrate tumour regression using the formula [1−(mean actual tumour weight_{FINAL(treated)}−mean tumour weight_{DAY 1(treated)}/mean actual tumour weight_{FINAL(vehicle control)}−mean tumour weight_{DAY 1(vehicle control)})×100%].

### Results

In these studies, “compound 1” was found to result in TGI of 34% at both doses tested.

### Example 4

### In Vitro Determination of Human Hepatocyte Microsomal Degradation Half-Life

### General Experimental Protocol

“Compound 1” (in 50% acetonitrile) was added to a microsomal incubation mixture (1:50 dilution) to achieve a final concentration that was less than the compound's solubility limit in pH 7.4 phosphate buffer,

the final concentration of ACN in the microsomal incubation was 1%,

samples were incubated in a water bath at 37° C., and

45 μL aliquots were taken over 2 hr, quenched with ACN, placed on ice for 30 min to precipitate proteins and assayed by LC/MS/MS using either the Quattro Ultima Pt, LCT or Q-T of instruments.

### Results

TABLE 3

| | Degradation | Predicted | Predicted | |
|---|---|---|---|---|
| | t_{1/2} | CLᵢₙₜ | CL_{blood} | Predicted |
| Compound | (min) | (mL/min/kg) | (mL/min/kg) | E_{H} |
| 1 | 32.2 | 62.2 | 15.5 | 0.75 |

### Example 5

### In Vitro Determination of Toxicity Using the ActiveTox® Suite of Assays

Compounds were analyzed in eight separate assays at concentrations of 10 and 100 μM in quadruplicate. Compounds were assayed tar toxicity via LDH release, inhibition of proliferation, ATP content, caspase 3/7 activation. Compounds were assayed for induction of cyp1A and P-glycoprotein inhibition. Compounds were also assayed for cyp3A induction under conditions which favor activation via the pregnane receptor (PXR) or the glucorticord receptor (GR). Appropriate positive and negative controls were included in each case. “Compound 1” showed no statistical effects in any of the above assays from the ActiveTox® suite.

### Example 6

### In Vitro Cell Proliferation Measurements as an Indicator of Compound Toxicity

A simple cell proliferation assay was used to determine the in vitro cytotoxicity of “compound 1.” Selected cell lines were cultured according to their specific requirements. The optimal cell density required for each cell line was determined. All compounds were tested at a single concentration in triplicate. Cell viability was determined using the CellTiter 96® AQueous One reagent from Promega Corporation.

Assays are performed by adding the test compound to the cells in culture and incubating the cells for a fixed period. The number of viable cells remaining after the incubation period is determined by adding a small amount of the CellTiter 96® AQueous One Solution Reagent directly to culture wells, incubating for 1-4 hours and then recording absorbance at 490 nm with a 96 well plate reader.

Controls in the assay include untreated cells, wells without cells and cells treated with know cytotoxic agents. Data is presented as % inhibition of cell proliferation. “Compound 1” showed no inhibitory effect on cell proliferation of 3T3, MCF_{—}7, or Jurkat cell lines at 100 μM.

### Example 7

### In Vitro Determination of Absorption of “Compound 1” by Determination of Transport across a Caco-2 Cell Monolayer

The Caco-2 assay was performed according to the procedure described in the following cited article [Caco-2 Monolayers in Experimental and Theoretical Predictions of Drug Transport, Artursson P, Palm K, Luthman K., Adv. Drug Deliv. Rev., 2001, 46, 27-43]. “Compound 1” was shown to have a Pₐₚₚ of 1.04×10⁻⁶ cm/sec relative to mannitol at 8.13×10⁻⁷ cm/sec.

### Example 8

### Solubility Data

TABLE 4
Solubility Data for Salts of “Compound 1”

| | Water | 5% Glucose |
|---|---|---|
| Salt | (mg/mL)^{a} | (mg/mL)^{b} |
| Free Base (Compound 1) | >0.2 | * |
| TFA | 3.64 | 5.27 |
| Citrate | 4.54 | * |
| Acetate | 6.96 | * |
| Methanesulphonate | 5.17 | * |
| Hydrochloride | 7.87 | 6.30 |
| Succinate | 8.52 | 4.98 |

*Not Examined
^{a}overnight
^{b}Day 3

### Example 9

### In Vitro Screening of Compounds against Somatostatin Subtypes SSTR-1 to SSTR-5

### General Method

Receptor membrane preparations containing the desired cloned receptor (for example cloned human somatostatin receptor subtype 5, SSTR5) and radio-labeled ligand (for example 3-[¹²⁵I]iodotyrosyl¹¹ Somatostatin-14)) were diluted at the concentration required for testing and according to the specific parameters associated with the selected receptor-ligand combination, including receptor Bₘₐₓ, ligand K_{d} and any other parameters necessary to optimize the experimental conditions. When tested for competition activity to the reference ligand, “compound 1” was mixed with membrane suspension and the radiolabeled reference ligand (with or without an excess of cold ligand to the receptor for determination of non-specific binding) and incubated at the temperature required by internal standard operating procedures. Following incubation, the binding reaction was stopped by the addition of ice-cold washing buffer and filtered on appropriate filters, which are then counted. Data analysis and curve-fitting was performed with XLfit (IDBS).

### Preparation of Compounds

10 mM solutions of test compounds in 100% DMSO were prepared. ˜160 μl was used for each dilution (20 μl/well in triplicate).

A 1.25 mM assay stock was prepared by making a 1:8 dilution of the 10 mM solution. (To 30 μL of the 10 mM solution was added 210 μL milli-Q H₂O. A 1:5 dilution series in milli-Q H₂O was then prepared.

| | | Final concentration | Final |
|---|---|---|---|
| | | concentration | concentration |
| | | in SST4 assay | in SST5 assay |
| A. | 240 μL of 1.25 mM | 0.25 mM | 0.125 mM |
| B. | 48 μL A + 192 μL mQ | 0.05 mM | 0.025 mM |
| C. | 24 μL B + 192 μL mQ | 0.01 mM | 0.005 mM |
| etc | | | |

Assays were performed in triplicate at each concentration within the 1:5 dilution series: 250 μM, 50 μM, 10 μM, 2 mM, 0.4 μM, 0.08 μM, 0.016 μM, 0.0032 μM, etc. (for SST4 assay) and 125 μM, 10 μM, 2 μM, 1 μM, 0.5 μM, etc (for SST5 assay).

### Filter Plate Assay for SST5 Receptor

Human SST5 somatostatin receptor was transfected into HEK-293 EBNA cells. Membranes were suspended in assay buffer (50 mM Tris-HCl, 1 mM EGTA, 5 mM MgCl₂, 10% sucrose, pH 7.5). The receptor concentration (Bₘₐₓ) was 0.57 pmol/mg proteinK_{d} for [¹²⁵I]SST-14 Binding 0.31 nM, volume 0.4 ml per vial (400 microassays/vial), and protein concentration 1.03 mg/ml.

After thawing the frozen receptor preparation rapidly, receptors were diluted with binding buffer, homogenized, and kept on ice.
1. Use Multiscreen glass fiber filter plates (Millipore, Cat No MAFCNOB10) precoated with o.5% PEI for ˜2 hr at 4° C. Before use add 200 μl/well assay buffer and filter using Multiscreen Separation System.
2. Incubate 5.5 μg of membranes (40 μl of a 1:40 dilution), buffer and [¹²⁵I]SST-14 (4 nM, ˜80 000 cpm, 2000 Ci/mmol) in a total volume of 200 μl for 60 min at 25° C. Calculate IC50 for SST-14 (a truncated version of the natural ligand SST-28) (Auspep, Cat No 2076) and SST-28 (Auspep, Cat No 1638). Prepare serial dilutions (1:5) of compounds, as described above and instead of adding SST-14 in well, add 20 μl of compounds (Table 1).
3. Filter using Multiscreen Separation System with 5×0.2 ml ice-cold Assay buffer.
4. Remove the plastic underdrain and dry plate in oven for 1 hr at 40° C.
5. Seal tape to the bottom of the plate.
6. Add 50 μl/well scintillant (Supermix, Wallac, Cat No 1200-439).
7. Seal and count in the BJET, program 2.

TABLE 5

| | | | Compounds |
|---|---|---|---|
| Volume (ul) | TB | NSB | testing |
| Membranes (5.5 μg/well) | 40 | 40 | 40 |
| Hot label (~80 000 cpm, ~4 nm) | 40 | 40 | 40 |
| Cold hormone | — | 20 | — |
| mQH₂O | 20 | — | — |
| Compounds | | | 20 |
| Assay buffer | 100 | 100 | 100 |
| Total volume (ul) | 200 | 200 | 200 |

TB: total binding
NSB: non-specific binding

In primary screening experiments compounds were tested in duplicate for a particular concentration. When determination of IC₅₀ values was required ten concentrations of the compounds were tested in duplicate such that the concentration range covered several log units above and below the expected IC₅₀.

Membranes were contacted with a test compound and a radioactive ligand (SST-14) versus a blank with radioactive ligand only and no test compound. The percentage of displaced radioactive ligand in the test samples relative to the blank, at two concentrations of each test compound, is represented as inhibition of binding in Table 6 below.

TABLE 6

| | |
|---|---|
| | Scaffold type A |
| 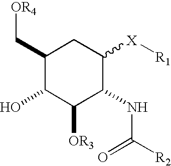 | |
| | Scaffold type B |
| 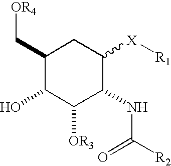 | |

Inhibition of binding of SST-14 to SSTR5

| | | | | | | | SSTR5 | |
|---|---|---|---|---|---|---|---|---|
| | | | | | | | Inhib | Inhib |
| | Scaffold | | | | | | 10 uM | 0.5 uM |
| # | Type | X | R1 | R2 | R3 | R4 | % | % |
| 17 | B | O | Me2Nap | PrG | MePh4Cl | Me | 98 | 84 |
| 18 | B | O | MePh | EtN | Me2Nap | Me | 98 | 90 |
| 19 | A | O | MePh4Cl | PrN | Me2Nap | Me | 98 | 88 |
| 20 | B | O | Me2Nap | PrN | MePh4Cl | Me | 97 | 76 |
| 21 | B | O | Me2Nap | EtN | MePh | Me | 97 | 81 |
| 22 | B | O | MePh | EtN | MePh4Cl | Me | 96 | 80 |
| 23 | A | O | Me2Nap | PrG | MePh4Cl | Me | 96 | 80 |
| 24 | B | O | MePh4Cl | PrN | Me2Nap | Me | 96 | 67 |
| 25 | A | O | Me2Nap | PrN | MePh4Ph | Me | 96 | 77 |
| 26 | B | O | Me2Nap | EtN | MePh4Ph | Me | 96 | 77 |
| 27 | B | O | MePh4Cl | PrN | MePh4Ph | Me | 96 | 74 |
| 28 | B | O | EtPh | PrN | Me2Nap | Me | 96 | 70 |
| 29 | A | O | Me2Nap | PrN | MePh4Ph | Me | 96 | 77 |
| 30 | A | O | MePh4Cl | PrN | MePh4Ph | Me | 96 | 80 |
| 31 | A | O | MePh4Ph | PrN | MePh4Ph | Me | 96 | 85 |
| 32 | A | O | MePh | PrN | Me2Nap | Me | 95 | 80 |
| 33 | A | O | MePh4Cl | PrN | MePh4Cl | Me | 95 | 68 |
| 34 | B | O | EtPh | EtN | MePh4Cl | Me | 95 | 61 |
| 35 | B | O | Me2Nap | EtG | MePh4Cl | Me | 95 | 60 |
| 36 | B | O | Me2Nap | EtG | MePh4Ph | Me | 95 | 67 |
| 37 | B | O | MePh | EtN | Me2Nap | Me | 95 | 81 |
| 38 | A | O | MePh4Ph | PrN | Me2Nap | Me | 95 | 79 |
| 39 | B | O | MePh4Cl | EtN | MePh4Cl | Me | 95 | 74 |
| 40 | A | O | MePh4Cl | PrN | Me | MePh4Ph | 95 | 66 |
| 41 | B | O | MePh4Cl | PrN | MePh4Cl | Me | 94 | 83 |
| 42 | A | O | EtPh | PrN | MePh4Cl | Me | 94 | 77 |
| 43 | B | O | Me2Nap | PrN | MePh4Ph | Me | 94 | 68 |
| 44 | A | O | EtPh | PrN | Me2Nap | Me | 94 | 78 |
| 45 | B | S | Me | MeG | MePh4Cl | MePh | 93 | 71 |
| 46 | B | O | MePh | PrN | MePh4Ph | Me | 93 | 71 |
| 47 | A | O | Me2Nap | PrN | MePh4Cl | Me | 93 | 68 |
| 48 | A | O | MePh4Cl | PrN | Me2Nap | Me | 92 | 66 |
| 49 | B | S | Me | PrG | Me2Nap | MePh4Cl | 92 | 60 |
| 50 | B | O | MePh4Cl | EtG | Me2Nap | Me | 92 | 74 |
| 51 | A | O | MePh4Cl | 2THPI | Me | Me2Nap | 92 | 76 |
| 52 | A | O | Me2Nap | EtG | MePh4Ph | Me | 92 | 69 |
| 53 | B | O | MePh | EtG | Me2Nap | Me | 91 | 58 |
| 54 | B | O | MePh | EtN | MePh4Ph | Me | 91 | 60 |
| 55 | A | O | MePh4Ph | PrG | MePh4Ph | Me | 91 | 71 |
| 56 | A | O | MePh4Cl | PrG | MePh4Cl | Me | 91 | 57 |
| 57 | B | O | Me2Nap | EtG | Me2Nap | Me | 91 | 64 |
| 58 | A | O | MePh4Ph | PrG | MePh4Ph | Me | 91 | 65 |
| 59 | B | O | MePh | PrN | MePh4Cl | Me | 90 | 66 |
| 60 | B | O | Me2Nap | PrG | Me2Nap | Me | 90 | 57 |
| 61 | A | O | Me2Nap | PrN | MePh4Cl | Me | 90 | 61 |
| 62 | B | O | EtPh | PrG | MePh4Cl | Me | 90 | 61 |
| 63 | A | O | MePh4Ph | PrN | MePh4Cl | Me | 90 | 50 |
| 64 | B | O | Me2Nap | PrG | MePh | Me | 90 | 57 |
| 65 | A | O | EtPh | PrN | MePh4Ph | Me | 90 | 61 |
| 66 | B | S | Me | EtN | Me2Nap | MePh4Ph | 89 | 61 |
| 67 | A | O | MePh4Cl | PrG | Me | MePh4Ph | 89 | 48 |
| 68 | A | O | Me2Nap | PrN | MePh | Me | 89 | 46 |
| 69 | B | O | MePh4Cl | 3PipG | Me | Me2Nap | 89 | 67 |
| 70 | A | O | MePh4Ph | MeG | Me | MePh4Ph | 89 | 18 |
| 71 | B | O | MePh4Cl | EtG | MePh4Cl | Me | 89 | 49 |
| 72 | B | S | Me | MeG | Me2Nap | MePh | 89 | 51 |
| 73 | A | O | EtPh | PrN | Me2Nap | Me | 89 | 56 |
| 74 | A | O | MePh4Ph | PrN | MePh4Cl | Me | 89 | 64 |
| 75 | A | O | Me2Nap | PrG | Me2Nap | Me | 89 | 61 |
| 76 | B | O | MePh | PrN | Me2Nap | Me | 88 | 51 |
| 77 | A | O | MePh4Ph | PrG | MePh4Ph | Me | 88 | 50 |
| 78 | A | O | MePh4Ph | EtG | Me | MePh4Cl | 88 | 43 |
| 79 | A | O | MePh4Cl | 4PipG | Me | Me2Nap | 88 | 55 |
| 80 | A | O | MePh4Ph | PrN | MePh4Ph | Me | 88 | 44 |
| 81 | A | O | Me2Nap | PrN | Me | Me2Nap | 87 | 60 |
| 82 | A | O | Me2Nap | PrN | MePh3OH | Me | 87 | 48 |
| 83 | B | S | Me | MeN | MePh4Ph | MePh4Cl | 87 | 61 |
| 84 | A | S | Me | Ph | MePh3N | MePh | 87 | 65 |
| 85 | B | O | Me2Nap | EtG | MePh | Me | 87 | 54 |
| 86 | A | O | Me2Nap | EtG | MePh4Ph | Me | 87 | 58 |
| 87 | B | O | MePh | EtG | MePh4Ph | Me | 86 | 43 |
| 88 | A | O | Me2Nap | MeG | MePh4Ph | Me | 86 | 63 |
| 89 | B | S | Me | EtG | MePh | Me2Nap | 86 | 58 |
| 90 | A | O | MePh4Cl | PrN | MePh4Ph | Me | 86 | 52 |
| 91 | B | S | Me | PrG | Me2Nap | MePh | 86 | 53 |
| 92 | B | O | Me2Nap | PrN | MePh | Me | 86 | 52 |
| 93 | A | O | MePh4Ph | PrG | MePh4Cl | Me | 86 | 47 |
| 94 | B | S | Me | PrN | MePh4Ph | MePh | 86 | 45 |
| 95 | A | O | MePh | PrN | MePh4Ph | Me | 85 | 44 |
| 96 | A | O | MePh4Cl | PrG | MePh4Ph | Me | 85 | 43 |
| 97 | B | S | Me | MeN | MePh4Ph | Me2Nap | 85 | 41 |
| 98 | A | O | EtPh | PrG | MePh4Cl | Me | 84 | 55 |
| 99 | B | O | MePh4Cl | EtG | MePh4Ph | Me | 84 | 43 |
| 100 | B | O | MePh | PrG | Me2Nap | Me | 84 | 59 |
| 101 | A | O | MePh4Cl | EtG | MePh4Ph | Me | 84 | 50 |
| 102 | B | O | MePh | EtG | MePh4Cl | Me | 84 | 42 |
| 103 | A | O | Me2Nap | PrG | MePh4Cl | Me | 84 | 48 |
| 104 | A | O | Me2Nap | PrG | Me2Nap | Me | 84 | 47 |
| 105 | B | S | Me | EtN | MePh | Me2Nap | 83 | 44 |
| 106 | B | O | EtPh | EtN | MePh | Me | 83 | 53 |
| 107 | A | O | MePh4Ph | PrG | Me2Nap | Me | 83 | 54 |
| 108 | A | O | MePh4Ph | EtG | MePh4Cl | Me | 83 | 47 |
| 109 | B | S | Me | EtN | Me2Nap | MePh | 83 | 44 |
| 110 | A | O | MePh | PrN | MePh4Cl | Me | 83 | 45 |
| 111 | B | S | Me | MeG | MePh4Ph | MePh4Cl | 83 | 36 |
| 112 | B | O | MePh | EtG | MePh | Me | 83 | 58 |
| 113 | A | O | MePh4Cl | PrN | MePh3OH | Me | 83 | 50 |
| 114 | B | S | Me | EtG | Me2Nap | Me2Nap | 82 | 47 |
| 115 | A | O | Me2Nap | EtG | Me | Me2Nap | 82 | 50 |
| 116 | B | S | Me | MeG | MePh4Cl | Me2Nap | 82 | 42 |
| 117 | B | O | MePh4Cl | PrN | MePh | Me | 82 | 46 |
| 118 | B | S | Me | EtG | MePh4Cl | MePh | 81 | 37 |
| 119 | B | O | MePh | MeG | MePh4Ph | Me | 81 | 37 |
| 120 | A | O | MePh4Ph | PrG | MePh4Cl | Me | 81 | 46 |
| 121 | A | O | MePh4Cl | PrN | Me | Me2Nap | 81 | 32 |
| 122 | A | O | MePh4Cl | PrN | Me | MePh4Cl | 81 | 39 |
| 123 | A | O | MePh4Cl | EtG | MePh4Cl | Me | 81 | 40 |
| 124 | B | H | — | EtN | MePh4Cl | MePh4Ph | 80 | 50 |
| 125 | B | S | Me | EtG | Me2Nap | MePh4Cl | 80 | 34 |
| 126 | A | O | Me2Nap | MeG | Me | Me2Nap | 80 | 57 |
| 127 | B | S | Me | PrN | Me2Nap | MePh4Ph | 80 | 48 |
| 128 | B | S | Me | EtG | Me2Nap | MePh | 80 | 28 |
| 129 | A | O | MePh4Ph | MeG | MePh4Ph | Me | 80 | 36 |
| 130 | A | O | MePh4Ph | PrG | MePh4Ph | Me | 80 | 40 |
| 131 | B | S | Me | MeG | Me2Nap | Me2Nap | 80 | 52 |
| 132 | A | O | EtPh | PrG | MePh4Ph | Me | 80 | 39 |
| 133 | B | O | Me2Nap | MeG | MePh4Cl | Me | 80 | 24 |
| 134 | A | O | Me2Nap | PrG | Me | MePh4Cl | 79 | 55 |
| 135 | A | O | Me2Nap | PrG | MePh | Me | 79 | 45 |
| 136 | A | O | MePh4Ph | EtG | Me | MePh4Ph | 79 | 26 |
| 137 | A | O | Me2Nap | MeG | Me2Nap | Me | 79 | 43 |
| 138 | B | O | MePh | PrG | MePh4Cl | Me | 79 | 39 |
| 139 | B | S | Me | MeG | Me2Nap | MePh4Ph | 79 | 34 |
| 140 | A | O | EtPh | PrN | Me | Me2Nap | 78 | 38 |
| 141 | A | O | EtPh | PrN | Me | MePh4Cl | 78 | 36 |
| 142 | B | S | Me | EtG | MePh | MePh4Cl | 78 | 44 |
| 143 | A | O | Me2Nap | PrN | Me | MePh4Cl | 78 | 35 |
| 144 | A | O | MePh | PrN | MePh3OH | Me | 78 | 45 |
| 145 | A | O | EtPh | PrN | MePh4Cl | Me | 78 | 45 |
| 146 | A | O | Me2Nap | PrN | MePh3OH | Me | 77 | 40 |
| 147 | A | O | EtPh | PrN | MePh3OH | Me | 77 | 40 |
| 148 | B | S | Me | PrN | MePh4Cl | MePh4Ph | 76 | 38 |
| 149 | B | S | Me | MeG | MePh4Ph | MePh | 76 | 35 |
| 150 | B | S | Me | MeG | Me2Nap | MePh4Cl | 76 | 42 |
| 151 | A | O | Me2Nap | EtG | Me2Nap | Me | 76 | 36 |
| 152 | A | O | MePh4Cl | EtG | Me | MePh4Cl | 76 | 47 |
| 153 | B | S | Me | PrN | Me2Nap | MePh4Cl | 76 | 34 |
| 154 | B | O | MePh | PrN | MePh | Me | 76 | 33 |
| 155 | B | S | Me | PrG | MePh4Ph | MePh | 76 | 46 |
| 156 | B | S | Me | PrN | Me2Nap | MePh | 76 | 25 |
| 157 | A | O | MePh4Ph | PrG | Me | MePh4Ph | 75 | 20 |
| 158 | B | S | Me | EtN | MePh4Cl | Me2Nap | 75 | 36 |
| 159 | B | O | MePh4Cl | MeG | Me2Nap | Me | 75 | 31 |
| 160 | A | O | MePh4Cl | PrG | Me2Nap | Me | 74 | 40 |
| 161 | A | O | Me2Nap | PrN | Me | MePh4Ph | 74 | 51 |
| 162 | B | O | MePh | MeG | Me2Nap | Me | 74 | 30 |
| 163 | B | O | MePh | EtN | MePh | Me | 73 | 34 |
| 164 | A | O | MePh4Cl | PrN | MePh | Me | 73 | 39 |
| 165 | A | O | MePh | PrG | MePh4Ph | Me | 73 | 37 |
| 166 | A | O | EtPh | EtG | MePh4Cl | Me | 73 | 36 |
| 167 | B | S | Me | MeN | Me2Nap | MePh4Cl | 73 | 43 |
| 168 | A | O | EtPh | PrG | MePh4Cl | Me | 72 | 47 |
| 169 | A | O | Me2Nap | EtG | Me | MePh4Ph | 72 | 27 |
| 170 | A | O | Me2Nap | EtG | MePh | Me | 72 | 29 |
| 171 | A | O | MePh | PrG | Me | MePh4Ph | 72 | 44 |
| 172 | A | O | MePh | EtG | MePh4Ph | Me | 72 | 21 |
| 173 | A | O | MePh4Cl | MeG | Me | Me2Nap | 71 | 47 |
| 174 | B | O | Me2Nap | MeG | Me2Nap | Me | 71 | 31 |
| 175 | A | N | 2Nap | PrG | MePh4Cl | Me | 71 | 29 |
| 176 | B | O | MePh4Cl | PrG | MePh | Me | 71 | 37 |
| 177 | A | O | MePh4Cl | MeG | Me | MePh4Ph | 71 | 47 |
| 178 | A | O | MePh4Ph | PrG | Me2Nap | Me | 71 | 39 |
| 179 | A | O | mePh4Ph | PrN | Me | MePh4Ph | 70 | 29 |

Where scaffold type A is of the D-gluco configuration as shown and type B is of the D-Allo configuration as shown. X may be either Sulfur (S), Oxygen (O) or an amide functionality (N) in which the nitrogen is bound to the anomeric position of the carbohydrate ring.

The substituents at R1, R3 and R4 are described as: Me is methyl (CH3); MePh is benzyl; MePh4Cl is p-chlorobenzyl; MePh4Ph is p-phenylbenzyl; Me2Nap is beta-napthylmethyl; MePh3OH is m-hydroxybenzyl; MePh3N is m-aminobenzyl; EtPh is phenethyl or ethylphenyl;

The substituents at R2 are described as: MeN is methylamino —CH₂—NH₂; EtN is ethylamino —CH₂—CH₂—NH₂; PrN is n-propylamino —CH₂—CH₂—CH₂—NH₂; MeG in methylguanidinium —CH₂—NH—C(═NH)—NH₂; EtG is ethylguanidinium —CH₂—CH₂—NH—C(═NH)—NH₂; PrG is propylguanidinium —CH₂—CH₂—CH₂—NH—C(═NH)—NH₂; 3-PipG is

TABLE 7

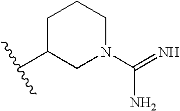

4-PipG is;

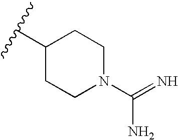

2THPI is

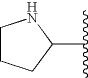

Binding of “Compound 1” to Somatostatin Receptor Subtypes 1-5

| | | | | |
|---|---|---|---|---|
| SSTR1 | SSTR2 | SSTR3 | SSTR4 | SSTR5 |

“Compound 1”

| | | | | | |
|---|---|---|---|---|---|
| IC50 | >12.5 μM | 7.5 μM | 8.5 μM | >12.5 μ | 322 nM |
| Hillslope | ND¹ | 1.2 | 1.04 | ND | 0.71 |
| selectivity | ND | 23.2 | 26.4 | ND | 1 |

SST-28 control

| | | | | | |
|---|---|---|---|---|---|
| IC50 | 5.137 nM | 1.09 nM | 2.49 nM | 12.074 nM | 0.66 nM |
| Hillslope | −1.42 | −2.03 | −1.49 | −1.23 | −1.5 |
| selectivity | 7.8 | 1.6 | 3.7 | 18 | 1 |

IC50 represents the concentration of compound required to displace 50% of the competitive radioligand.
Selectivity is the normalized IC50: that is the lowest IC50 for a compound is assigned a value of 1 and each other IC50 is some multiple of that number.
SST-28 is the natural ligand and is a positive control for this experiment.

TABLE 8
Kᵢ Values for Compounds 2-16

| Compound | SSTR1 | SSTR2 | SSTR3 | SSTR4 | SSTR5 |
|---|---|---|---|---|---|
| 2 | 2280 | 728 | 203 | 3258 | 353 |
| 3 | 3732 | 1230 | NoModel | 4578 | NoModel |
| | | | (127) | | (147) |
| 4 | 1595 | 842 | 188 | 1424 | 530 |
| 5 | 2547 | 1572 | 285 | 8542 | 153 |
| 6 | >10000 | NoModel | NoModel | >10000 | 151 |
| | | (29247) | (206) | | |
| 7 | 6092 | 498 | 131 | 9133 | 22 |
| 8 | 3092 | 2009 | 599 | 2774 | 598 |
| 9 | >10000 | 490 | NoModel | 463 | NoModel |
| | | | (0.8) | | (1.1) |
| 10 | >10000 | 726 | NoModel | noModel | 220 |
| | | | (228) | | |
| 11 | 9221 | 4846 | NoModel | 3984 | NoModel |
| | | | (2) | | (3) |
| 12 | >10000 | 5060 | NoModel | >10000 | 46 |
| | | | (429) | | |
| 13 | >10000 | >10000 | NoModel | >10000 | 62 |
| | | | (476) | | |
| 14 | >10000 | 1210 | 340 | >10000 | 549 |
| 15 | 1956 | 1038 | 792 | 2257 | 73 |
| 16 | 2369 | 860 | 441 | 4378 | 85 |

Kᵢ is expressed in nM concentration. Where Kᵢ could not be determined, the corresponding IC50 in nanomolar concentration is shown in parentheses. Kᵢ is defined by the Michaelis-Menten kinetic equation as described in “Biochemistry” by A. Lehninger.
No Model indicates the slope of the curve is such that a reasonable Ki could not be extracted.

### Example 10

### Endothelial Cell Proliferation Assays

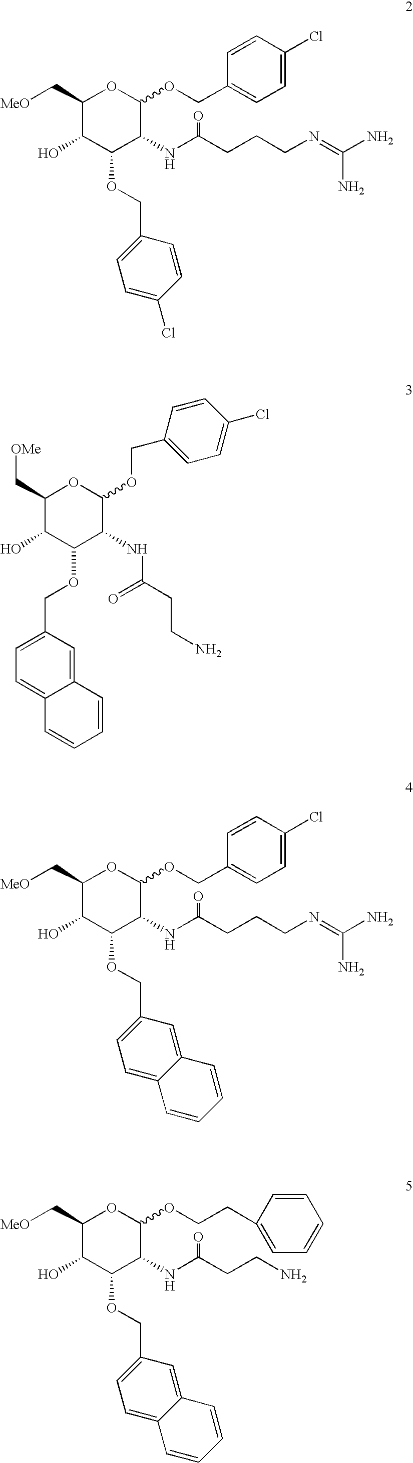

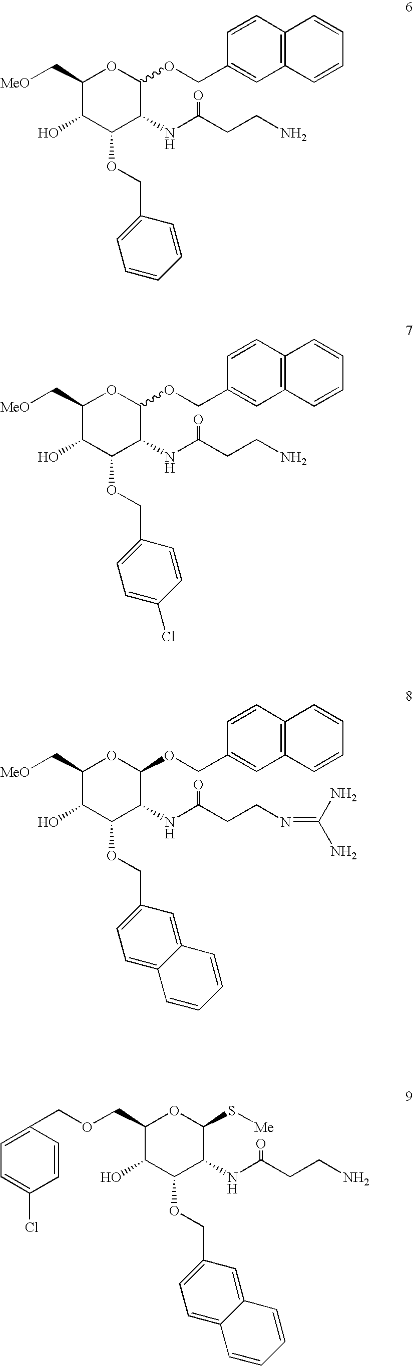

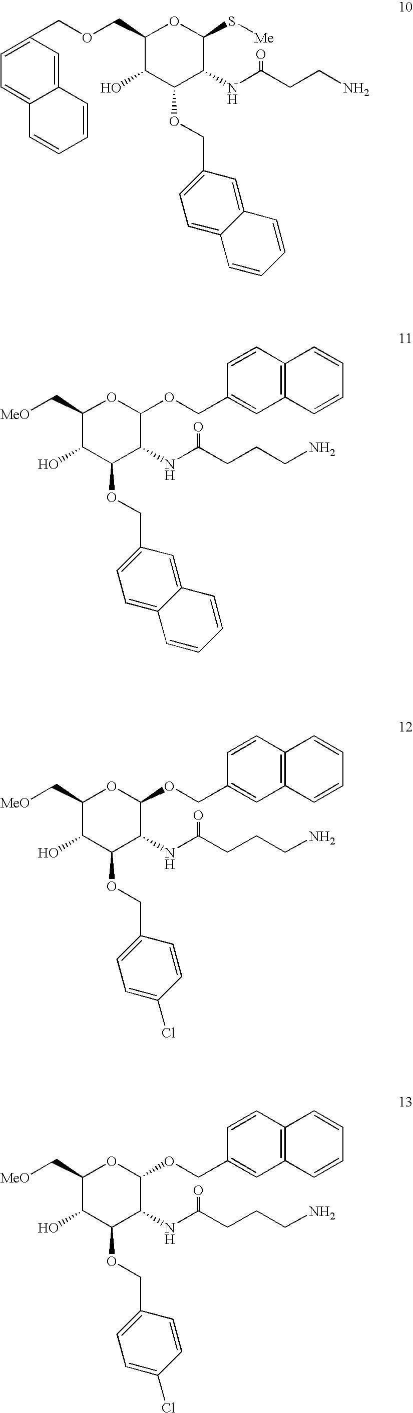

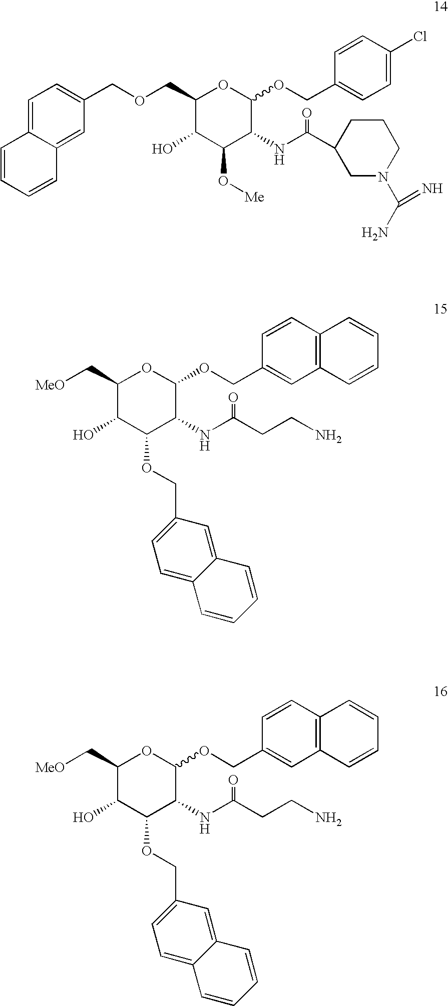

Human Umbilical Vein Endothelial Cells (HUVEC from Clonetics) were plated in 96 well plates at 1000 cell per well in EGM-2 medium (Clonetics). Cells were grown overnight at 37° C. in 5% CO₂. Fresh EGM-2 medium containing compound at the desired concentration was added to the wells and the cells allowed to grow for a further 48 hrs. A MTS (Promega) colorimetric assay was performed after 48 hrs to determine cell growth according to the manufacturers instructions. Results are presented in terms of percentage growth.

TABLE 9

% Growth

| Compound Number | 125 uM | 250 uM |
|---|---|---|
| 2 | −83 | −91 |
| 3 | −75 | −39 |
| 4 | −98 | −90 |
| 5 | −89 | −57 |
| 6 | 24 | −78 |
| 7 | −81 | −63 |
| 8 | 66 | −18 |
| 9 | −86 | −78 |
| 10 | −85 | −63 |
| 11 | 29 | −53 |
| 12 | −95 | −71 |
| 13 | 58 | −80 |
| 14 | −95 | −90 |
| 15 | −78 | −67 |
| 1 | 69 | 17 |

### Example 11

### In Vivo Efficacy Evaluation of “Compound 1” in a Nude Mouse Model of Human MV522 Non-Small Cell Lung Cancer (NSCLC) Tumours

Initial Maximum Tolerated Dose (MTD) studies were done in male nude mice to determine the appropriate intravenous dosing regimen for the MV522 human NSCLC tumour model. A range of doses between 0 and 50 mg/Kg for 28 days (qdx28) were tested. Mice were randomized into groups with 5 animals per group including vehicle control. Animals were weighed twice weekly starting on day one and observed daily for adverse reactions or toxicity due to the agent. MTD studies determined the selection of 20 mg/kg and 35 mg/kg for intravenous dosing once per day for 28 days in the MV522 human NSCLC tumour xenograft model.

Male nude mice (nu/nu) between 5 and 6 weeks of age weighing approximately 20 g were implanted subcutaneously (s.c) by trocar with fragments of MV522 human tumour carcinomas harvested from s.c growing tumours in host mice. MV-522 is a metastatic human lung adenocarcinoma line. When tumours reached approximately 72 mg in size animals were pair matched into treatment and control groups with 10 mice in each group. Each mouse was tagged and followed individually throughout the experiment.

“Compound 1” was administered i.v. in a saline vehicle from day one. Vehicle control group animals were administered saline i.v. There were nine treatment groups including the vehicle control group. Group one animals received saline, i.v. qdx28; group 2 received “compound 1” 20 mg/kg, i.v. qdx28; group 3 received “compound 1” 35 mg/kg i.v. qdx28; group four received paclitaxel 8 mg/kg i.p. qdx5; group five received paclitaxel 16 mg/kg i.p. qdx5; group six received 20 mg/kg of “compound 1” i.v. qdx28 and paclitaxel 8 mg/kg imp. qdx5; group seven received 20 mg/kg of “compound 1” i.v. qdx28 and paclitaxel 16 mg/kg i.p. qdx5; group eight received 35 mg/kg of “compound 1” i.v. qdx28 and paclitaxel 8 mg/kg i.p. qdx5; group nine received 35 mg/kg of “compound 1” i.v. qdx28 and paclitaxel 16 mg/kg i.p. qdx5.

Mice were weighed twice weekly and tumour measurements were obtained using calipers twice weekly. Collection of measurements started on day 1. Tumour measurements were converted into tumour volume (mm³) using the standard formula [(W²×L)/2].

At the end of the treatment period the mice were weighed and sacrificed. Each tumour was excised and weighed and a mean actual tumour weight (mg) per group was calculated along with the mean actual volume (mm³). Mice having a tumour with less volume than on day 1 were classified as having partial tumour regression. Mean tumour regression was determined using the formula [1−(mean actual tumour weight_{FINAL}/mean tumour weight_{DAY 1})×100%].

Tumour growth inhibition (TGI) was calculated for each group containing treated animals that did not demonstrate tumour regression using the formula [1−(mean actual tumour weight_{FINAL(treated)}−mean tumour weight_{DAY1(treated)}/mean actual tumour weight_{FINAL(vehicle control)}−mean tumour weight_{DAY1(vehicle control)})×100%].

### Results

In these studies TGI was found in group five (42.1%), group seamen (56.9%) and group nine (75.1%)

### Example 12

### Tube Formation Assays

HUVEC (Clonetics) were plated in 96 well plates in EGM-2 media (Clonetics) at 2.5 to 3×10⁴ cells per well. Cells of less than 6 passages were used in all studies. Wells were precoated with 50 μL growth factor containing Matrigel (Becton Dickinson). HUVEC were allowed to form tubes by incubation at 37° C. in 5% CO₂ for 22 hrs. In test wells compound was added in the desired concentration. Photographic images of the wells ×4 magnification were used to determine size of the tubes, length of tubes and number of junctions using image analysis software. This data was used to determine if matrigel tube formation was disrupted in the presence of test compounds and in some cases to calculate the concentration of compound that resulted in 50% inhibition of tube formation (EC₅₀).

TABLE 10
Matrigel tube formation disruption

| Compound Number | 125 uM | 250 uM |
|---|---|---|
| 2 | | Y |
| 3 | Y | Y |
| 4 | | Y |
| 5 | | Y |
| 6 | | Y |
| 7 | Y | Y |
| 16 | Y | Y |
| 8 | | Y |
| 9 | Y | Y |
| 10 | Y | Y |
| 11 | | Y |
| 12 | | Y |
| 13 | | Y |
| 14 | Y | Y |
| 15 | Y | Y |
| 1 | | Y |

TABLE 11
IC50 determination of selected compounds from above set.

| Compound Number | EC50 in Matrigel assay | |
|---|---|---|
| 1 | 250 | uM |
| 7 beta | 95 | uM |
| 7 alpha | 50 | uM |
| 3 beta | 90 | uM |
| 3 alpha | 45 | uM |
| 16 alpha | 55 | uM |
| 10 | 125 | uM |
| 9 | 50 | uM |
| 14 mixture | 155 | uM |
| 14 single anomer | 155 | uM |

### Example 13

### Preparation of “Compound 1”

Methods that can be used in the preparation of “compound 1” are described in Alchemia patent application PCT AU03/01008 (WO 2004/014929) which is hereby incorporated by reference to this application.

The preparation of “compound 1” is also described in scheme 1 below. Thus, in a typical experiment, D-GluNAc (115, 15 g) was suspended in 2-phenylethanol (375 ml) and heated at 120° C. Acetyl chloride (3.7 ml) was added and the mixture refluxed for ˜3 hrs. The reaction mixture was cooled to RT and poured into ether (2 L) to ppt A 146 g (86%). A (142.99 g) in acetonitrile (574 mL) was treated with p-methoxybenzaldehyde dimethylacetal (112 mL) and N-camphor sulphonic acid (2.26 g) at 60° C. for 5 hrs. The mixture was cooled to RT and treated with Et3N (˜8 mL) and evaporated to dryness to give a brown solid. The solid was triturated with petrol to give a yellow brown solid and filtered to give B (211 g). B (203.26 g) was treated with diethylene glycol methyl ether (800 mL) and a solution of KOH (257 g) and heated at 120° C. for 5 hrs, cooled, then poured into water (4 L) and extracted with chloroform (4 L and 2 L). The combined organics where washed with brine (2 L) and the organics evaporated to dryness. The residue was stirred with water (4 L), filtered and washed with water (500 mL). The solid product was dried under high vacuum and azeotroped with toluene until dry to give C (189 g). C (89.5 g) in MeOH (560 mL) was treated with a solution of CuSO4.5H2O (1.32 g) in water (2 mL) with stirring. Triflic azide was added and the mixture stirred at room temperature for 4 hr. The reaction is then quenched with n-butylamine (15 mL) and was filtered through a celite pad and evaporated. The residue was dissolved in EtOH/water (5:1) and cooled to 0° C. The product was filtered, washed and dried to give D (141.4 g). To a stirred suspension of 60% NaH (8.42 g; 0.21 mol) in dry DMF (100 mL) was added a solution of D (59.67 g) in dry DMF (100 mL) over 30 min and then stirred at 0° C. for a further 30 min, 2-(bromomethyl)naphthalene (37.09 g) in dry DMF (50 mL) was then added drop wise over 30 mins and then stirred for a further 1 hr. The reaction was quenched with methanol (10 mL) until no further hydrogen evolution and the mixture evaporated to dryness under high vacuum. The crude residue was dissolved in DCM (1000 mL) and washed with water (500 mL), 10% HCl (500 mL), NaHCO3 (500 mL) and brine (5 L). The organic layer was evaporated and azeotroped with toluene to give dark yellow solid. The residue was dissolved in EtOAc (300 mL) and precipitated with petroleum ether (4 L) to give E. (40.3%).

To a solution of E (34.6 g) in DCM (350 mL) at 0-5° C., was added 1M BH3 in THF (91.53 g; 91.53 mmol, 1.5 eq) over 30 min. After an additional 15 nm in of stirring, Bu2BOTf (6.10 mL; 6.1 mmol; 0.1 eq) was added slowly to the mixture. The ice bath was removed and the reaction stirred at RT for 2 hr. The reaction was quenched by slow addition of MeOH and the diluted with DCM (1500 mL). The organic mixture was washed with sat. NaHCO3 (200 mL), dried MgSO4 and evaporated to give a glassy solid. The crude product was chromatographed with Tol:EtOAc eluant mixes from silica to yield F (23.3 g). F (20 g; 35.15 mmol) dissolved in dry DMF (55 mL) and cooled in an ice bath (0-5° C.) and treated with NaH (2.0 eq). Methyl iodide (3.32 mL) was added drop wise and stirred at RT for 4 hr. The reaction quenched with methanol and diluted with DCM (2 L) and washed with water (1 L. The aqueous layer wash back extracted with DCM (2×100 mL). The combined organics were dried (MgSO4) and evaporated to give G (19.6 g). G (23.4 g) in a mixture of DMF (54.6 mL) and MeOH (155 mL) was treated with a solution of NH4Cl (10.73 g), followed by activated zinc dust (13.1 g) and stirred at RT for 1 hr. The mixture was diluted with DCM (2 L) and filtered through a celite pad. The DCM layer seas washed with sat NaHCO3 solution (1 L), dried MgSO4 and evaporated to give H (20.7 g). To a solution of 4-Boc-NH-butyric acid (11.974 g) in dry DMF was added HBTU (21.465 g) and DIPEA (8.783 g) with stirring at room temperature. The resulting solution was stirred at RT for 20 min and then added to a solution of H (25.24 g) in DMF (200 mL). The reaction was stirred at RT for 1 hr and quenched with 1N NaOH (40 mL). The reaction was diluted with DCM (2 L) and washed with 0.5N NaOH (800 mL). The organic layer was dried (MgSO4) and evaporated to dryness to give I (30.8 g). I (15 g) in dry DCM (200 mL) was treated with Et3SiH (20 mL) and then TFA (25 mL) and stirred at RT for 2 hr. The reaction was evaporated to dryness (water bath at 25° C.) and the residue taken up into DCM (500 mL) and basified with 25% NH4OH solution (90 mL) and again evaporated to dryness. The residue was dissolved in DCM (500 mL) and washed with water (200 mL) and 1N NaOH (100 mL). The organic layer was washed with brine (100 mL), dried MgSO4 and evaporated to give J (8.1 g).

### Example 14

### Preparation of Compounds 2-179

Methods suitable for the preparation of compounds 2 to 179 are described in Alchemia patent application PCT AU03/01008 (WO 2004/014929) which is hereby incorporated by reference to this application. The materials used in these studies were prepared by the solid phase methods described in PCT AU03/01008.

TABLE 12
Mass Spectral Data for Compounds 1-16

| compound | M + H observed | M+ Calculated |
|---|---|---|
| 1 | 523.321 | |
| 2 | 570.071 | 568.19 |
| 3 | 529.099 | 528.2 |
| 4 | 586.151 | 584.24 |
| 5 | 510.03 | 508.26 |
| 6 | 495.192 | 494.24 |
| 7 | 529.277 | 528.2 |
| 8 | 587.33 | 586.28 |
| 9 | 545.209 | 544.18 |
| 10 | 561.311 | 560.23 |
| 11 | 559.322 | 558.27 |
| 12 | 543.34 | 542.22 |
| 13 | 543.34 | 542.22 |
| 14 | 611.359 | 610.26 |
| 15 | 545.31 | 544.26 |
| 16 | 545.121 | 544.26 |

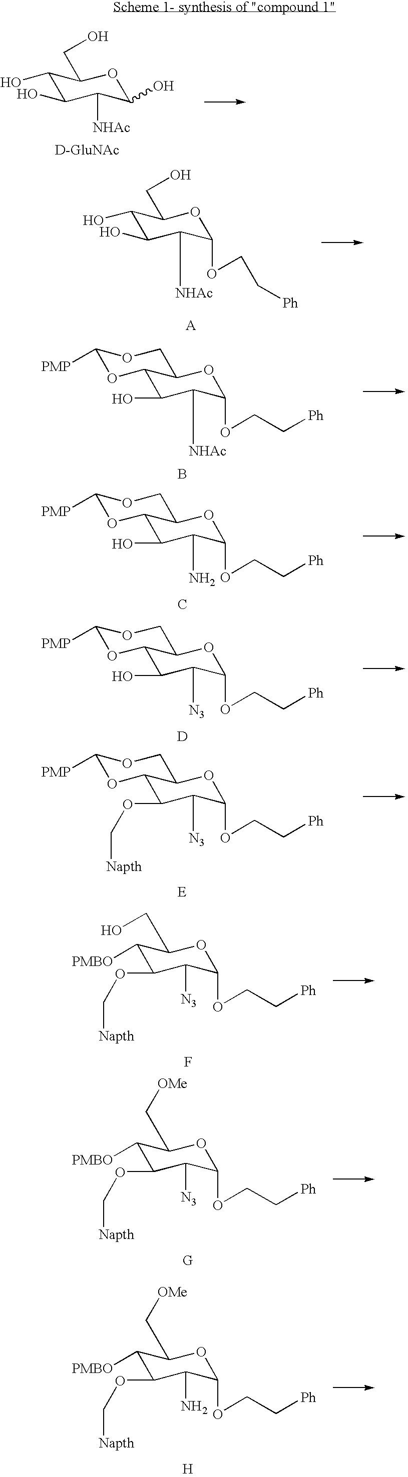

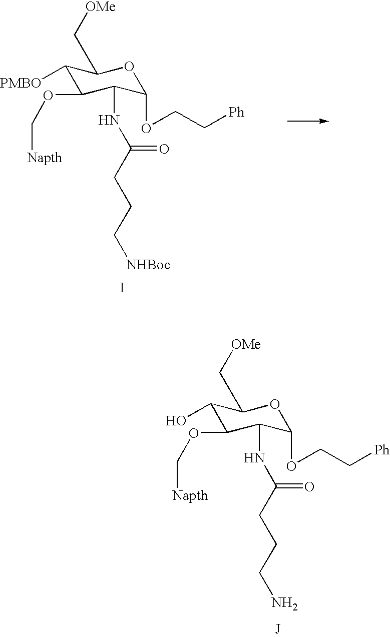

Throughout the specification and the claims (if present), unless the context requires otherwise, the term “comprise”, or variations such as “comprises” or “comprising,” will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents and patent applications cited herein are hereby incorporated by reference in their entirety for all purposes to the same extent as if each individual publication, patent or patent application were specifically and individually indicated to be so incorporated by reference.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

1: A method for inhibiting angiogenesis in a subject comprising administering to the subject in need thereof an effective amount of at least one compound of General Formula I 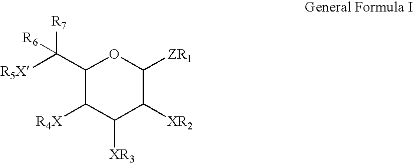
wherein the ring or any chiral center(s) may be of any configuration;
Z is sulphur, oxygen, CH₂, C(O), C(O)HN, NH, NR^{A} or hydrogen, in the case where Z is hydrogen then R₁ is not present;
R^{A} is selected from the set defined for R₁ to R₅;
X and X′ are independently oxygen or nitrogen providing that at least one X of General Formula I is nitrogen, X or X′ may also combine independently with one of R₁ to R₅ to form an azide;
R₁ to R₅ are independently selected from the group consisting of H or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear; and
R₆ and R₇ are hydrogen, or may combine to form a carbonyl function;
or a tautomer, ester, solvate or pharmaceutically acceptable salt thereof.

2: The method of claim 1, wherein angiogenesis is inhibited by contacting a receptor associated with angiogenesis with at least one said compound.

3: The method of claim 2, wherein the receptor is a somatostatin receptor.

4: The method of claim 3, wherein the receptor is somatostatin receptor subtype 5.

5: The method of claim 1, wherein the at least one compound is a compound of General Formula II 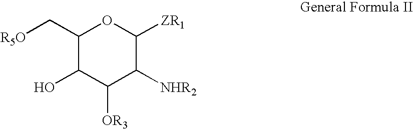
wherein R₁, R₂, R₃, R₅, and Z are defined as in claim 1, or a tautomer, ester, solvate or pharmaceutically acceptable salt thereof.

6: The method of claim 1, wherein the at least one compound comprises a compound of General Formula III 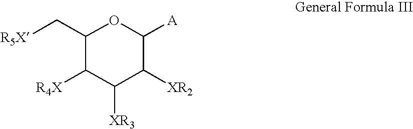
wherein A is defined as hydrogen, SR₁, or OR₁, where R₁ is defined as in claim 1, and
wherein X, X′, R₂, R₃, R₄, and R₅ are defined as in claim 1, or a tautomer, ester, solvate or pharmaceutically acceptable salt thereof.

7: The method of claim 1, wherein the at least one compound is a compound of General Formula IV 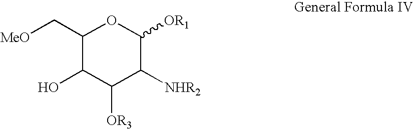
wherein R₁, R₂, and R₃ are defined as in claim 1,
or a tautomer, ester, solvate or pharmaceutically acceptable salt thereof.

8: The method of claim 1, wherein the at least one compound is a compound of General Formula V 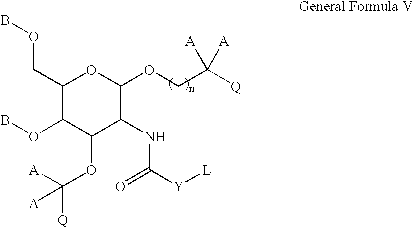
wherein the stereochemistry may be alpha or beta at the anomeric carbon, and may be axial or equatorial at the other pyranosyl ring carbons;
n is 0 or 1;
‘Y’ is selected from substituted or unsubstituted C₁-C₈ alkyl, hetero alkyl, cyclo-alkyl, aromatic or heterocyclic spacer, where the substituents selected from the group consisting of nitro, chloro, fluoro, bromo, nitrile, carboxyl, —NH₂, —NHR, —NHB, C₁₋₃ alkyl, —OR, azido, —C(O)NH₂, —C(O)NHR, —C(O)N(R)₂, —N(R)C(O)R, —N(H)C(O)R, —CF₃, and —SR,
wherein R is independently selected from a substituted or unsubstituted alkyl, aryl and heterocyclic group;
L is selected from —NB₂, and guanidinium,
wherein B is defined as below, and, additionally ‘Y’ and ‘L’ can combine to form a substituted or unsubstituted nitrogen containing heterocycles;
Q are independently selected from a substituted or unsubstituted monocyclic or bicyclic aromatic or heteroaromatic, where the substituents are defined as for ‘Y’;
A are independently selected from hydrogen, chloro, fluoro and methyl; and
B are independently selected from H, methyl, ethyl, and propyl,
or a tautomer, ester, solvate or pharmaceutically acceptable salt thereof.

9: The method of claim 8, wherein the at least one compound is a compound of General Formula VI 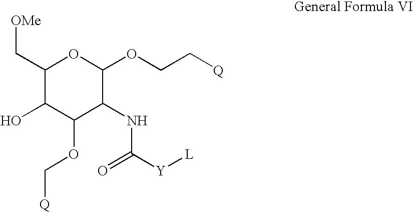
where Y, L, and Q are as defined in claim 8,
or a tautomer, ester, solvate or pharmaceutically acceptable salt thereof.

10: The method of claim 8, wherein the at least one compound is a compound of General Formula VII 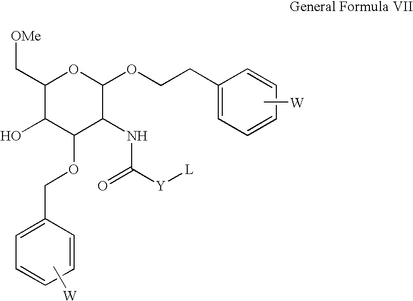
wherein ‘W’ is mono-, di-, tri-, or tetrasubstitution and ‘W’ may be the same or different, or ‘W,’ in combination with the aromatic ring, is a substituted or unsubstituted fused ring system, which is hetero-atomic or homo-atomic, and may be aromatic or aliphatic,
wherein the substituents are selected from the group consisting of phenyl, C₁₋₄ alkyl, heterocycles, nitro, chloro, fluoro, bromo, nitrile, carboxyl, —NH₂, —NHR, —NR₂, C₁₋₃ alkyl, —OR, azido, —C(O)NH₂, —C(O)NHR, —C(O)N(R)₂, —N(R)C(O)R, —N(H)C(O)R, —CF₃, and —SR, wherein R is independently selected from a substituted or unsubstituted alkyl, aryl or heterocyclic group;
and where Y and L are as defined in claim 8,
or a tautomer ester, solvate or pharmaceutically acceptable salt thereof.

11: The method of claim 10, wherein the at least one compound is a compound of General Formula VIII 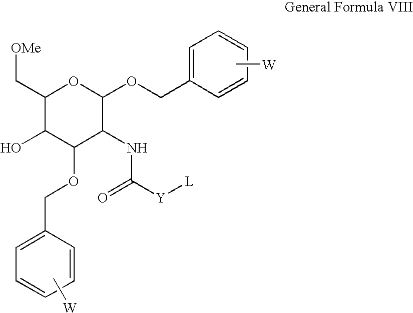
wherein, W, L and Y are as defined in claim 10,
or a tautomer, ester solvate or pharmaceutically acceptable salt thereof.

12: The method of claim 10, wherein the at least one compound is a compound of General Formula IX 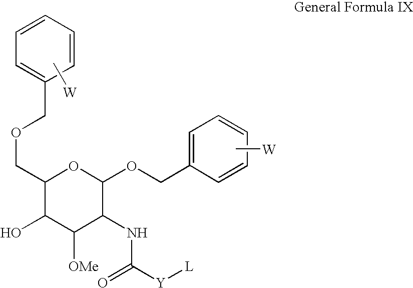
wherein, W, L and Y are as defined in claim 10,
or a tautomer, ester, solvate or pharmaceutically acceptable salt thereof.

13: The method of claim 10, wherein the at least one compound is a compound of General Formula X 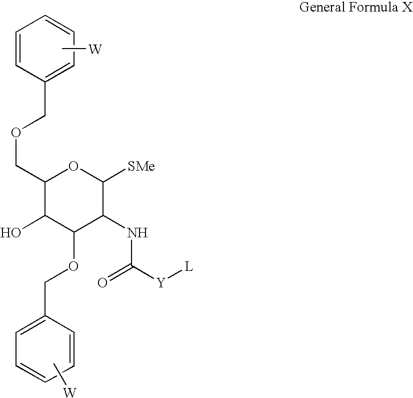
wherein, W, L and Y are as defined in claim 10,
or a tautomer, ester, solvate or pharmaceutically acceptable salt thereof.

14: The method of claim 1, wherein the optional substituents of R₁ to R₅ are independently selected from the group consisting of OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl and thioheteroaryl, any of which said substituents may optionally be further substituted with at least one moiety of the group consisting of OH, NO, NO₂, NH₂, N₃, halogen, CF₃, CHF₂, CH₂F, nitrile alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl and thioheteroaryl.

15: The method of claim 1, wherein the at least one compound is 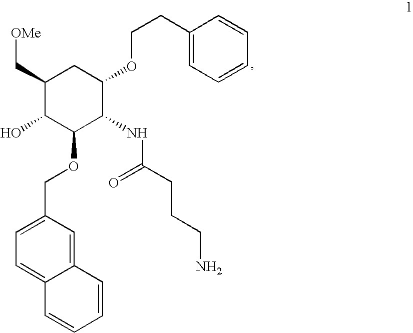
or a tautomer, ester, solvate or pharmaceutically acceptable salt thereof.

16: The method of claim 1, wherein the at least one compound is 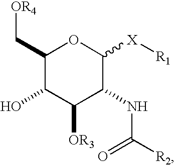
which is in the D-gluco configuration,
wherein
X is Sulfur (S), Oxygen (O) or an amide functionality (N) in which the nitrogen is bound to the anomeric position of the carbohydrate ring;
R₁, R₃ and R₄ are selected from the group consisting of methyl, benzyl, p-chlorobenzyl, p-phenylbenzyl, beta-napthylmethyl, m-hydroxybenzyl, m-aminobenzyl, phenethyl and ethylphenyl;
R₂ is selected from the group consisting of methylamino —CH₂—NH₂, ethylamino —CH₂—CH₂—NH₂, n-propylamino —CH₂—CH₂—CH₂—NH₂, methylguanidinium —CH₂—NH—C(═NH)—NH₂, ethylguanidinium —CH₂—CH₂—NH—C(═NH)—NH₂, propylguanidinium —CH₂—CH₂—CH₂—NH—C(═NH)—NH₂, 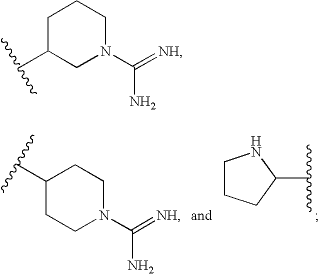
or a tautomer, ester, solvate or pharmaceutically acceptable salt thereof.

17: The method of claim 1, wherein the at least one compound is 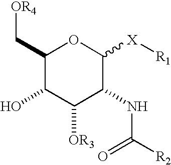
which is of the D-Allo configuration,
wherein
X is Sulfur (S), Oxygen (O) or an amide functionality (N) in which the nitrogen is bound to the anomeric position of the carbohydrate ring,
R1, R3 and R4 are selected from the group consisting of methyl, benzyl, p-chlorobenzyl, p-phenylbenzyl, beta-napthylmethyl, m-hydroxybenzyl, m-aminobenzyl, phenethyl and ethylphenyl,
R₂ is selected from the group consisting of methylamino —CH₂—NH₂, ethylamino —CH₂—CH₂—NH₂, n-propylamino —CH₂—CH₂—CH₂—NH₂, methylguanidinium —CH₂—NH—C(═NH)—NH₂, ethylguanidinium —CH₂—CH₂—NH—C(═NH)—NH₂, propylguanidinium —CH₂—CH₂—CH₂—NH—C(═NH)—NH₂, and 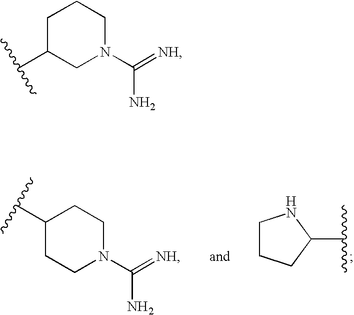
or a tautomer, ester, solvate or pharmaceutically acceptable salt thereof.

18: The method of claim 1, wherein the at least one compound is 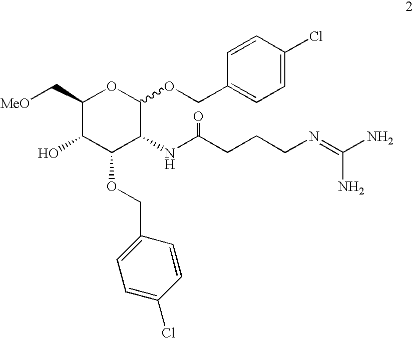
or a tautomer, ester, solvate or pharmaceutically acceptable salt thereof.

19: The method of claim 1, wherein the at least one compound is 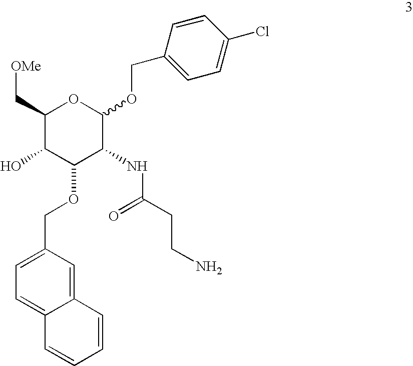
or a tautomer, ester, solvate or pharmaceutically acceptable salt thereof.

20: The method of claim 1, wherein the at least one compound is 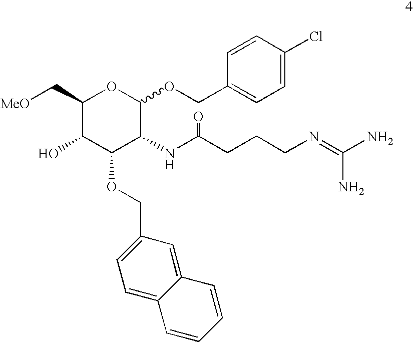
or a tautomer, ester, solvate or pharmaceutically acceptable salt thereof.

21: The method of claim 1, wherein the at least one compound is 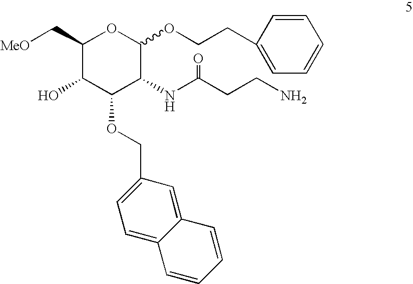
or a tautomer, ester, solvate or pharmaceutically acceptable salt thereof.

22: The method of claim 1, wherein the at least one compound is 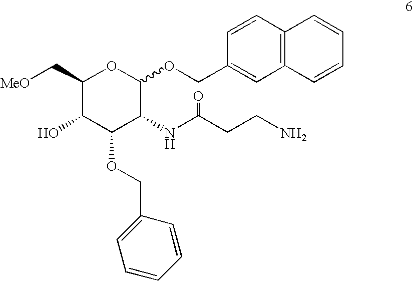
or a tautomer, ester, solvate or pharmaceutically acceptable salt thereof.

23: The method of claim 1, wherein the at least one compound is 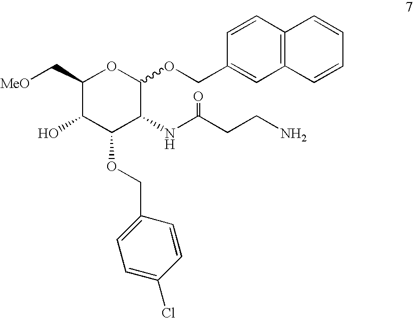
or a tautomer, ester, solvate or pharmaceutically acceptable salt thereof.

24: The method of claim 1, wherein the at least one compound is 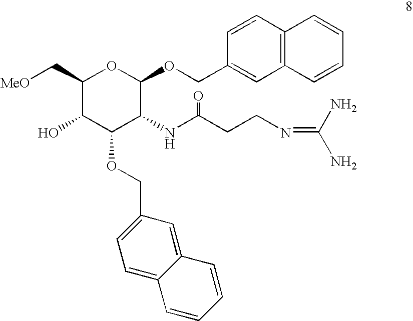
or a tautomer, ester, solvate or pharmaceutically acceptable salt thereof.

25: The method of claim 1, wherein the at least one compound is 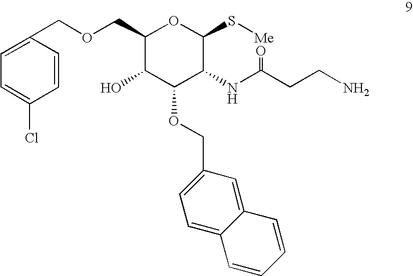
or a tautomer, ester, solvate or pharmaceutically acceptable salt thereof.

26: The method of claim 1, wherein the at least one compound is 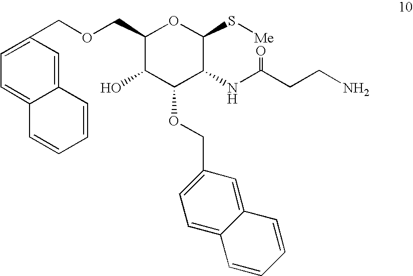
or a tautomer, ester, solvate or pharmaceutically acceptable salt thereof.

27: The method of claim 1, wherein the at least one compound is 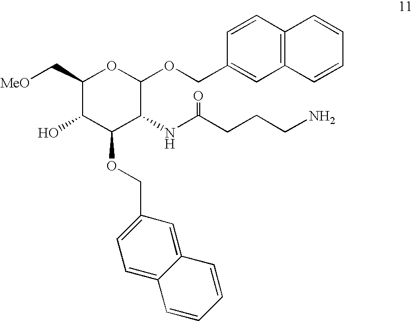
or a tautomer, ester, solvate or pharmaceutically acceptable salt thereof.

28: The method of claim 1, wherein the at least one compound is 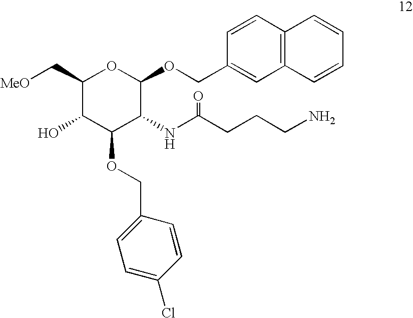
or a tautomer, ester, solvate or pharmaceutically acceptable salt thereof.

29: The method of claim 1, wherein the at least one compound is 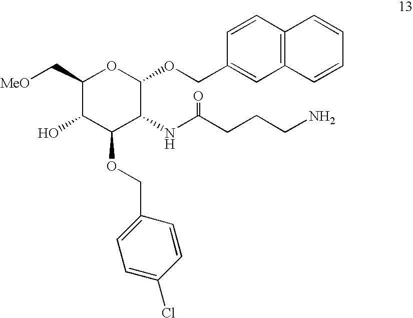
or a tautomer, ester, solvate or pharmaceutically acceptable salt thereof.

30: The method of claim 1, wherein the at least one compound is 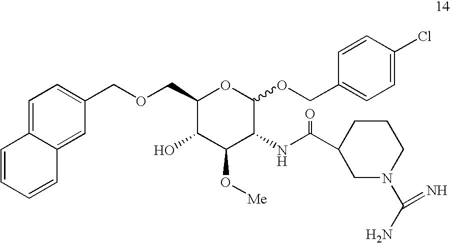
or a tautomer, ester, solvate or pharmaceutically acceptable salt thereof.

31: The method of claim 1, wherein the at least one compound is 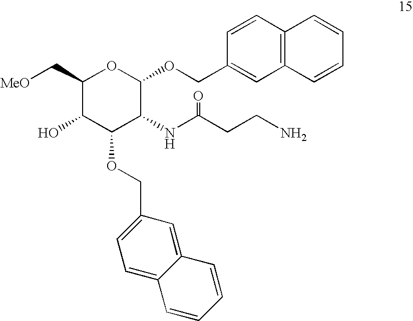
or a tautomer, ester, solvate or pharmaceutically acceptable salt thereof.

32: The method of claim 1, wherein the at least one compound is 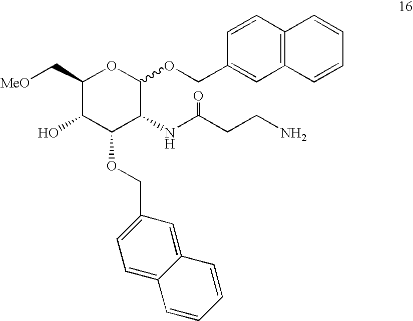
or a tautomer, ester, solvate or pharmaceutically acceptable salt thereof.

33. (canceled)

34: A method for inhibiting angiogenesis, comprising contacting a sample comprising a blood vessel or a cell associated with formation of blood vessels with an effective amount of at least one compound of General Formula I, 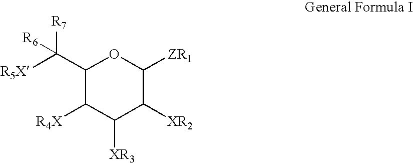
wherein the ring or any chiral center(s) may be of any configuration;
Z is sulphur, oxygen, CH₂, C(O), C(O)HN, NH, NR^{A} or hydrogen, in the case where Z is hydrogen then R₁ is not present;
R^{A} is selected from the set defined for R₁ to R₅;
X and X′ are independently oxygen or nitrogen providing that at least one X of General Formula I is nitrogen, X or X′ may also combine independently with one of R₁ to R₅ to form an azide;
R₁ to R₅ are independently selected from the group consisting of H or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear; and
R₆ and R₇ are hydrogen, or may combine to form a carbonyl function;
or a tautomer, ester, solvate or pharmaceutically acceptable salt thereof;
wherein contacting of said blood vessel or cell with said at least one compound inhibits angiogenesis.

35-36. (canceled)

37: A method for inhibiting angiogenesis, comprising contacting somatostatin receptor with an effective amount of at least one compound of General Formula I 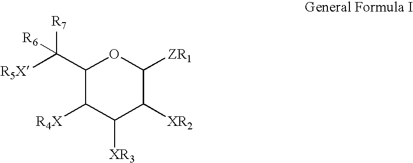
wherein the ring or any chiral center(s) may be of any configuration;
Z is sulphur, oxygen, CH₂, C(O), C(O)HN, NH, NR^{A} or hydrogen, in the case where Z is hydrogen then R₁ is not present;
R^{A} is selected from the set defined for R₁ to R₅;
X and X′ are independently oxygen or nitrogen providing that at least one X of General Formula I is nitrogen, X or X′ may also combine independently with one of R₁ to R₅ to form an azide;
R₁ to R₅ are independently selected from the group consisting of H or an alkyl, acyl, alkenyl, alkynyl, heteroalkyl, aryl, heteroaryl, arylalkyl or heteroarylalkyl substituent of 1 to 20 atoms, which is optionally substituted, and can be branched or linear; and
R₆ and R₇ are hydrogen, or may combine to form a carbonyl function;
or a tautomer, ester, solvate or pharmaceutically acceptable salt thereof;
wherein binding of said somatostatin receptor with said at least one compound inhibits angiogenesis.
